(19) United States
(12) Patent Application Publication
(10) Pub. No.: US 2024/0069645 A1
(43) Pub. Date: Feb. 29, 2024

(54) GESTURE RECOGNITION WITH HEALTHCARE QUESTIONNAIRES

(71) Applicant: atai Life Sciences AG, Berlin (DE)

(72) Inventor: Paul Rollinger, San Diego, CA (US)

(21) Appl. No.: 18/228,885

(22) Filed: Aug. 1, 2023

Related U.S. Application Data
(60) Provisional application No. 63/394,393, filed on Aug. 2, 2022.

(51) Int. Cl. G06F 3/01; G16H 10/20

(52) U.S. Cl. CPC G06F 3/017; G16H 10/20

(57) ABSTRACT

A method of presenting a digital questionnaire, comprising: receiving, at a user's device, a digital questionnaire including a plurality of questions; presenting, by the user's device, a first virtual page; processing a first gesture received while the first virtual page is being presented; presenting, by the user's device, a second virtual page, wherein the second virtual page is selected based on the first gesture; processing, a second gesture received while the second virtual page is being presented; presenting a third virtual page; processing, a third gesture received while the third virtual page is being presented.

### CROSS REFERENCE TO RELATED APPLICATIONS

This application claims priority to U.S. Patent Application Ser. No. 63/394,393, filed on Aug. 2, 2022, the entirety of which is herein incorporated by reference.

### BACKGROUND

Drug therapies have been used to treat many different types of medical conditions and disorders. Drug therapies can be administered to a patient to target a specific condition or disorder. Examples of suitable drug therapies can include pharmaceutical medications, biological products, etc. Treatments for certain types of mood and/or substantive use disorders can also involve counseling sessions, psychotherapy, or other types of structured interactions. As part of a patient's treatment, a patient may be asked to provide information as part of a questionnaire.

### SUMMARY

According to embodiments, a method of presenting and processing a digital questionnaire by a system including a server and a user's device executing an application, wherein the user's device includes an interactive display, includes: transmitting, from the server, data corresponding to the digital questionnaire to the user's device, wherein the digital questionnaire includes a plurality of virtual pages; receiving, at the user's device, the data corresponding to the digital questionnaire from the server; processing, by the application running on the user's device, the data corresponding to the digital questionnaire; causing, by the application running on the user's device, data for a first one of the virtual pages to be presented on the interactive display of the user's device; processing a first input signal corresponding to a first user input on the interactive display, wherein the first input signal is generated at least in part while the first one of the virtual pages is presented on the interactive display; determining whether the first input signal includes data corresponding to a first gesture, wherein the first gesture is received at the interactive display; determining a character of the first gesture; assigning a first value corresponding to the first gesture; assigning the first value as a response to a first question on the first one of the virtual pages; causing, by the application running on the user's device, data for a second one of the virtual pages to be presented on the interactive display of the user's device; processing a second input signal corresponding to a second user input on the interactive display, wherein the second input signal is generated at least in part while the second one of the virtual pages is presented on the interactive display; determining the second input signal includes data corresponding to a second gesture, wherein the second gesture is received at the interactive display; determining a character of the second gesture; assigning a second value corresponding to the second gesture; assigning the second value as a response to a second question on the second one of the virtual pages; causing, by the application running on the user's device, data for a third one of the virtual pages to be presented on the interactive display of the user's device; processing a third input signal corresponding to a third user input on the interactive display, wherein the third input signal is generated at least in part while the third one of the virtual pages is presented on the interactive display; determining whether the third input signal includes data corresponding to a third gesture, wherein the third gesture is received at the interactive display; determining a character of the third gesture; and assigning a third value corresponding to the second gesture. The character of at least one of the first gesture, the second gesture, or the third gesture may include a swipe. The at least one of the first value, the second value, or the third value may include one of a binary value. At least one of the data for the second one of the virtual pages or the data for the third one of the virtual pages may be selected based at least in part on at least one of the first value or the second value. The method may further include: causing, by the application running on the user's device, data for a fourth one of the virtual pages to be presented on the interactive display of the user's device; and processing a fourth input signal corresponding to a fourth user input on the interactive display, wherein the fourth input signal is generated at least in part while the fourth one of the virtual pages is presented on the interactive display, wherein the fourth input signal does not include data corresponding to a gesture. The method may further include assessing at least one additional data during at least a portion of a time period between said causing data for the first one of the virtual pages to be presented on the interactive display of the user's device and said processing the first input signal corresponding to the first user input on the interactive display. The at least one additional data may include at least one of a duration of the time period, a signal from a camera of the user's device, or biometric data.

According to embodiments, a system for presenting a digital questionnaire to a user includes: a user's device; and a server, wherein: the server is configured to transmit data corresponding to the digital questionnaire to the user's device, wherein the digital questionnaire includes a plurality of virtual pages; the user's device is configured to receive the data corresponding to the digital questionnaire; the user's device is configured to process the data corresponding to the digital questionnaire; the user's device is configured to cause data for a first one of the virtual pages to be presented on the interactive display of the user's device; at least one of the server or the user's device is configured to process a first input signal corresponding to a first user input on the interactive display, wherein the first input signal is generated at least in part while the first one of the virtual pages is presented on the interactive display; at least one of the server or the user's device is configured to determine whether the first input signal includes data corresponding to a first gesture, wherein the first gesture is received at the interactive display; at least one of the server or the user's device is configured to determine a character of the first gesture; at least one of the server or the user's device is configured to assign a first value corresponding to the first gesture; at least one of the server or the user's device is configured to assign the first value as a response to a first question on the first one of the virtual pages; the user's device is configured to case data for a second one of the virtual pages to be presented on the interactive display of the user's device; at least one of the server or the user's device is configured to process a second input signal corresponding to a second user input on the interactive display, wherein the second input signal is generated at least in part while the second one of the virtual pages is presented on the interactive display; at least one of the server or the user's device is configured to determine the second input signal includes data corresponding to a second gesture, wherein the second gesture is received at the interactive display; at least one of the server or the user's device is configured to determine a character of the second gesture; at least one of the server or the user's device is configured to assign a second value corresponding to the second gesture; at least one of the server or the user's device is configured to assign the second value as a response to a second question on the second one of the virtual pages; the user's device is configured to cause data for a third one of the virtual pages to be presented on the interactive display of the user's device; at least one of the server or the user's device is configured to process a third input signal corresponding to a third user input on the interactive display, wherein the third input signal is generated at least in part while the third one of the virtual pages is presented on the interactive display; at least one of the server or the user's device is configured to determine whether the third input signal includes data corresponding to a third gesture, wherein the third gesture is received at the interactive display; at least one of the server or the user's device is configured to determine a character of the third gesture; and at least one of the server or the user's device is configured to assign a third value corresponding to the second gesture. The character of at least one of the first gesture, the second gesture, or the third gesture may include a swipe. At least one of the first value, the second value, or the third value may include one of a binary value. At least one of the data for the second one of the virtual pages or the data for the third one of the virtual pages may be selected based at least in part on at least one of the first value or the second value. The user's device may be configured to cause data for a fourth one of the virtual pages to be presented on the interactive display of the user's device, at least one of the server or the user's device may be configured to process a fourth input signal corresponding to a fourth user input on the interactive display, wherein the fourth input signal is generated at least in part while the fourth one of the virtual pages is presented on the interactive display, and wherein the fourth input signal does not include data corresponding to a gesture. At least one of the server or the user's device may be configured to assess at least one additional data during at least a portion of a time period between said causing data for the first one of the virtual pages to be presented on the interactive display of the user's device and said processing the first input signal corresponding to the first user input on the interactive display. The at least one additional data may include at least one of a duration of the time period, a signal from a camera of the user's device, or biometric data.

According to embodiments, a non-transitory computer-readable storage medium has instructions that, when executed by at least one processor, cause the at least one processor to: transmit, from a server, data corresponding to a digital questionnaire to a user's device, wherein the digital questionnaire includes a plurality of virtual pages; receive, at the user's device, the data corresponding to the digital questionnaire from the server; process, by the user's device, the data corresponding to the digital questionnaire; cause, by the user's device, data for a first one of the virtual pages to be presented on the interactive display of the user's device; process a first input signal corresponding to a first user input on the interactive display, wherein the first input signal is generated at least in part while the first one of the virtual pages is presented on the interactive display; determine whether the first input signal includes data corresponding to a first gesture, wherein the first gesture is received at the interactive display; determine a character of the first gesture; assign a first value corresponding to the first gesture; assign the first value as a response to a first question on the first one of the virtual pages; cause, by the application running on the user's device, data for a second one of the virtual pages to be presented on the interactive display of the user's device; process a second input signal corresponding to a second user input on the interactive display, wherein the second input signal is generated at least in part while the second one of the virtual pages is presented on the interactive display; determine the second input signal includes data corresponding to a second gesture, wherein the second gesture is received at the interactive display; determine a character of the second gesture; assign a second value corresponding to the second gesture; assign the second value as a response to a second question on the second one of the virtual pages; cause, by the application running on the user's device, data for a third one of the virtual pages to be presented on the interactive display of the user's device; process a third input signal corresponding to a third user input on the interactive display, wherein the third input signal is generated at least in part while the third one of the virtual pages is presented on the interactive display; determine whether the third input signal includes data corresponding to a third gesture, wherein the third gesture is received at the interactive display; determine a character of the third gesture; and assigning a third value corresponding to the second gesture. The character of at least one of the first gesture, the second gesture, or the third gesture may include a swipe. The at least one of the first value, the second value, or the third value may include one of a binary value. The at least one of the data for the second one of the virtual pages or the data for the third one of the virtual pages may be selected based at least in part on at least one of the first value or the second value. The non-transitory computer-readable storage medium may include instructions that, when executed by at least one processor, further cause the at least one processor to: cause data for a fourth one of the virtual pages to be presented on the interactive display of the user's device; and process a fourth input signal corresponding to a fourth user input on the interactive display, wherein the fourth input signal is generated at least in part while the fourth one of the virtual pages is presented on the interactive display, wherein the fourth input signal does not include data corresponding to a gesture. The non-transitory computer-readable storage medium may include instructions for assessing at least one additional data during at least a portion of a time period between said causing data for the first one of the virtual pages to be presented on the interactive display of the user's device and said processing the first input signal corresponding to the first user input on the interactive display.

### BRIEF DESCRIPTION OF SEVERAL VIEWS OF THE DRAWINGS

FIG. 1 is a schematic block diagram of a system for treating a patient, according to an embodiment.

FIG. 2 is a schematic block diagram of a system for treating a patient including a mobile device and server for implementing digital therapy and/or monitoring and collecting information regarding a subject, according to an embodiment.

FIG. 3 is a data flow diagram illustrating information exchanged between different components of a system for treating a patient, according to an embodiment.

FIG. 4 is a flow chart illustrating a method of onboarding a new patient into a treatment protocol, according to an embodiment.

FIG. 5 is a flow chart illustrating a method of delivering assignments to a patient, according to an embodiment.

FIG. 6 is a flow chart illustrating a method of analyzing data collected from a patient, according to an embodiment.

FIG. 7 is a flow chart illustrating a method of analyzing data collected from a patient, according to an embodiment.

FIG. 8 is a flow chart illustrating an example of content being presented on a user device, according to an embodiment.

FIG. 9 illustrates an example schematic diagram illustrating a system of information exchange between a server and a user device (e.g., an electronic device), according to some embodiments.

FIG. 10 illustrates an example schematic diagram illustrating an electronic device implemented as a mobile device including a haptic subsystem, according to some embodiments.

FIG. 11 illustrates a flow chart of a process for providing feedback to a user in a digital questionnaire, according to some embodiments.

FIG. 12 shows examples of haptic effect patterns, according to some embodiments.

FIG. 13 shows an example user interface of the user device, according to some embodiments.

FIG. 14 is an example answer format having multiple axes, according to some embodiments.

FIG. 15 schematically depicts axes representing changes in one or more characteristics associated with an example haptic effect, according to some embodiments.

FIGS. 16A, 16B, and 16C show an example user interface of the user device, according to some embodiments.

The foregoing summary, as well as the following detailed description of certain techniques of the present application, will be better understood when read in conjunction with the appended drawings. For the purposes of illustration, certain techniques are shown in the drawings. It should be understood, however, that the claims are not limited to the arrangements and instrumentality shown in the attached drawings.

### DETAILED DESCRIPTION

The embodiments described herein relate to methods and systems for interacting with patients to receive information in a questionnaire, such as a questionnaire used as part of drug and/or counseling therapies.

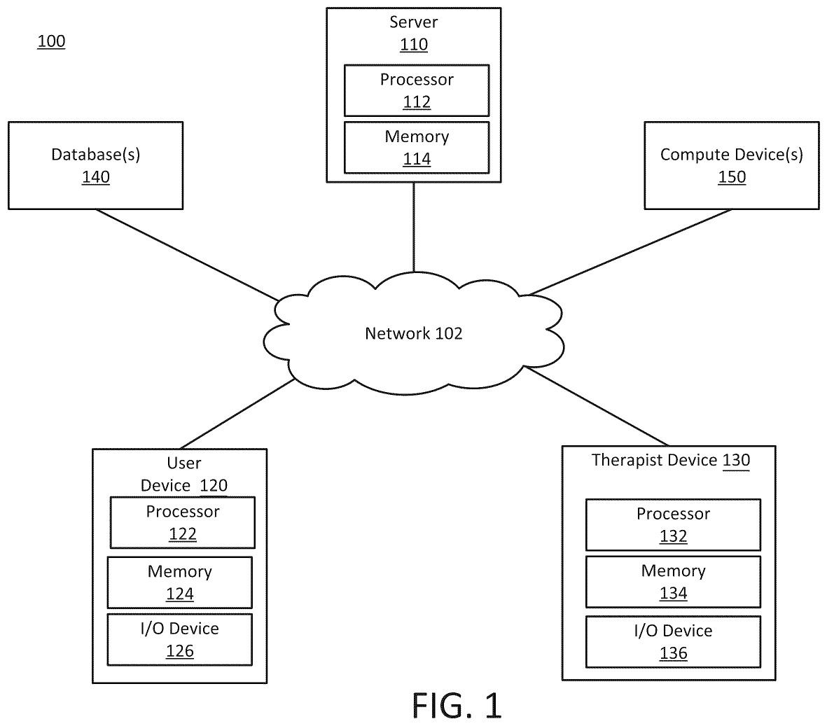

FIG. 1 depicts an example system, according to embodiments described herein. System 100 may be configured to provide digital content to patients and/or monitor and analyze information about patients. System 100 may be implemented as a single device, or be implemented across multiple devices that are connected to a network 102. For example, system 100 may include one or more compute devices, including a server 110, a user device 120, a therapy provider device 130, database(s) 140, or other compute device(s) 150. Compute devices may include component(s) that are distributed or integrated.

The server 110 may include component(s) that are remotely situated from other compute devices and/or located on premises near the compute devices. The server 110 can be a compute device (or multiple compute devices) having a processor 112 and a memory 114 operatively coupled to the processor 112. In some instances, the server 110 can be any combination of hardware-based modules (e.g., a field-programmable gate array (FPGA), an application specific integrated circuit (ASIC), a digital signal processor (DSP)) and/or software-based modules (computer code stored in memory 114 and/or executed at the processor 112) capable of performing one or more specific functions associated with that module. In some instances, the server 110 can be a server such as, for example, a web server, an application server, a proxy server, a telnet server, a file transfer protocol (FTP) server, a mail server, a list server, a collaboration server and/or the like. In some instances, the server 110 can include or be communicatively coupled to a personal computing device such as a desktop computer, a laptop computer, a smart phone, a personal digital assistant (PDA), a standard mobile telephone, a tablet personal computer (PC), and/or so forth.

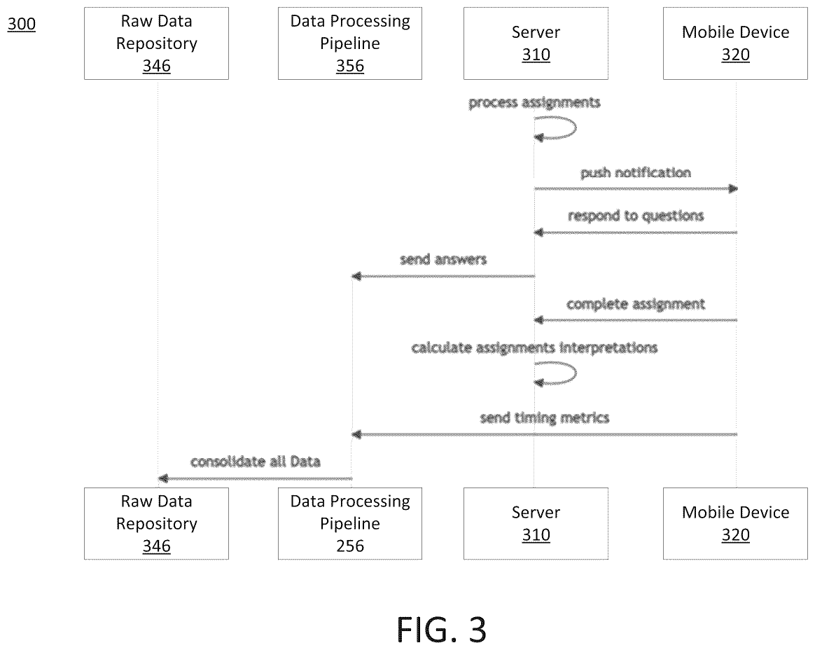

The memory 114 can be, for example, a random-access memory (RAM) (e.g., a dynamic RAM, a static RAM), a flash memory, a removable memory, a hard drive, a database and/or so forth. In some implementations, the memory 114 can include (or store), for example, a database, process, application, virtual machine, and/or other software code and/or modules (stored and/or executing in hardware) and/or hardware devices and/or modules configured to execute one or more processes, as described with reference to FIGS. 3-7 and 16A-16C. In such implementations, instructions for executing such processes can be stored within the memory 114 and executed at the processor 112. In some implementations, the memory 112 can store content (e.g., text, audio, video, or interactive activities), patient data, and/or the like.

The processor 112 can be configured to, for example, write data into and/or read data from the memory 114, and execute the instructions stored within the memory 114. The processor 112 can also be configured to execute and/or control, for example, the operations of other components of the server 110 (such as a network interface card, other peripheral processing components (not shown)). In some implementations, based on the instructions stored within the memory 114, the processor 112 can be configured to execute one or more steps of the processes depicted in FIGS. 3-7 and 16A-16C.

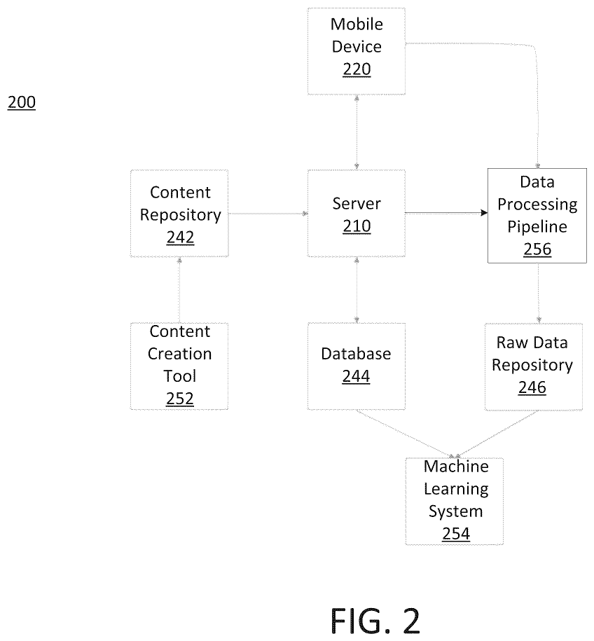

In some embodiments, the server 110 can be communicatively coupled to one or more database(s) 140. The database(s) 140 can include one or more repositories, storage devices and/or memory for storing information from patients, physicians and therapists, caretakers, and/or other individual involved in assisting and/or administering therapy and/or care to a patient. In some embodiments, the server 100 can be coupled to a first database for storing patient information and/or assignments (e.g., content, coursework, etc.) and a second database for storing chat and/or voice data received from the patient (e.g., responses to assignments, vocal-acoustic data, etc.). Further details of example database(s) are described with reference to FIG. 2.

The user device 120 can be a compute device associated with a user, such as a patient or a supporter (e.g., caretaker or other individual providing support or caring for a patient). The user device 120 can have a processor 122 and a memory 124 operatively coupled to the processor 122. In some instances, the user device 120 can be a cellular telephone (e.g., smartphone), tablet computer, laptop computer, desktop computer, portable media player, wearable digital device (e.g., digital glasses, wristband, wristwatch, brooch, armbands, virtual reality/augmented reality headset), and the like. The user device 120 can be any combination of hardware-based device and/or module (e.g., a field-programmable gate array (FPGA), an application specific integrated circuit (ASIC), a digital signal processor (DSP)) and/or software-based code and/or module (computer code stored in memory 122 and/or executed at the processor 121) capable of performing one or more specific functions associated with that module.

The memory 124 can be, for example, a random-access memory (RAM) (e.g., a dynamic RAM, a static RAM), a flash memory, a removable memory, a hard drive, a database and/or so forth. In some implementations, the memory 124 can include (or store), for example, a database, process, application, virtual machine, and/or other software code or modules (stored and/or executing in hardware) and/or hardware devices and/or modules configured to execute one or more processes as described with regards to FIGS. 3-7 and 16A-16C. In such implementations, instructions for executing such processes can be stored within the memory 124 and executed at the processor 122. In some implementations, the memory 124 can store content (e.g., text, audio, video, or interactive activities), patient data, and/or the like.

The processor 122 can be configured to, for example, write data into and/or read data from the memory 124, and execute the instructions stored within the memory 124. The processor 122 can also be configured to execute and/or control, for example, the operations of other components of the user device 120 (such as a network interface card, other peripheral processing components (not shown)). In some implementations, based on the instructions stored within the memory 124, the processor 122 can be configured to execute one or more steps of the processes described with respect to FIGS. 3-7 and 16A-16C. In some implementations, the processor 122 and the processor 112 can be collectively configured to execute the processes described with respect to FIGS. 3-7 and 16A-16C.

The user device 120 can include an input/output (I/O) device 126 (e.g., a display, a speaker, a tactile output device, a keyboard, a mouse, a microphone, a touchscreen, etc.), which can include a user interface, e.g., a graphical user interface, that presents information (e.g., content) to a user and receives inputs from the user. In some embodiments, the user device 120 can implement a mobile application that presents the user interface to a user. In some embodiments, the user interface can present content, including, for example, text, audio, video, and interactive activities, to a user, e.g., for educating a user regarding a disorder, therapy program, and/or treatment, or for obtaining information about the user in relation to a treatment or therapy program. In some embodiments, the content can be provided during a digital therapy session, e.g., for treating a medical condition of a patient and/or preparing a patient for treatment or therapy. In some embodiments, the content can be provided as part of a periodic (e.g., a daily, weekly, or monthly) check-in, whereby a patient is asked to provide information regarding a mental and/or physical state of the patient.

In some embodiments, the user device 120 may include or be coupled to one or more sensors (not shown in FIG. 1). For example, sensor(s) may be any suitable component that enables any of the compute devices described herein to capture information about a patient, the environment and/or objects in the environment around the compute device and/or convey information about or to a patient or user. Sensor(s) may include, for example, image capture devices (e.g., cameras), ambient light sensor, audio devices (e.g., microphones), light sensors, proprioceptive sensors, position sensors, tactile sensors, force or torque sensors, temperature sensors, pressure sensors, motion sensors, sound detectors, gyroscope, accelerometer, blood oxygen sensor, combinations thereof, and the like. In some embodiments, sensor(s) may include haptic sensors, e.g., components that may convey forces, vibrations, touch, and other non-visual information to compute device. In some embodiments, the user device 120 may be configured to measure one or more of motion data, mobile device data (e.g., digital exhaust, metadata, device use data), wearable device data, geolocation data, sound data, camera data, therapy session data, medical record data, input data, environmental data, social application usage data, attention data, activity data, sleep data, nutrition data, menstrual cycle data, cardiac data, voice data, social functioning data, or facial expression data.

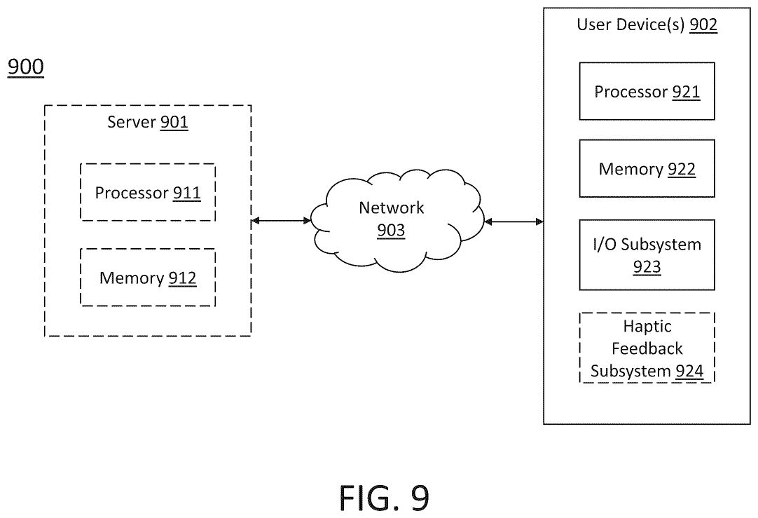

In some embodiments, the user device 120 may be configured to track one or more of a user's responses to interactive questionnaires and surveys, diary entries and/or other logging, vocal-acoustic data, digital biomarker data, and the like. For example, the user device 120 may present one or more questionnaires or exercises for the patient to complete. As used herein, a “questionnaire” includes a survey, exercise, or any presentation of information intended to solicit a response from a user. Further, a “digital questionnaire” includes a questionnaire presented by a computing device, such as user device 120. Unless specified or is otherwise clear from the context, any reference to a questionnaire herein is to a digital questionnaire. In some implementations, the user device 120 can collect data during the completion of the questionnaire or exercise. Results may be made available to a therapist and/or physician. In some embodiments, when a user provides input into the user device 120, the device can generate and use haptic feedback (e.g., vibration) to interact with the patient. The vibration can be in different patterns in different situations, as described with reference to FIGS. 9-15.

In some embodiments, the user device 120 and/or the server 110 (or other compute device) coupled to the user device 120 can be configured to process and/or analyze the data from the patient and evaluate information regarding the patient, e.g., whether the patient has a particular disorder, whether the patient has increased brain plasticity and/or motivation for change, etc. Based on the analysis, certain information can be provided to a therapist and/or physician, e.g., via the therapy provider device 130.

The therapy provider device 130 may refer to any device configured to be operated by one or more providers, healthcare professionals, therapists, caretakers, etc. Similar to the user device 120, the therapy provider device 130 can include a processor 132, a memory 134, and an I/O device 136. The therapy provider device 130 can be configured to receive information from other compute devices connected to the network 102, including, for example, information regarding patients, alerts, etc. In some embodiments, therapy provider device 130 can receive information from a provider, e.g., via I/O device 136, and provide that information to one or more other compute devices. For example, a therapist during a therapy session can input information regarding a patient into the therapy provider device 130 via I/O device 136, and such information can be consolidated with other information regarding the patient at one or more other compute devices, e.g., server 110, user device 120, etc. In some embodiments, the therapy provider device 130 can be configured to control content that is delivered to a patient (e.g., via user device 120), information that is collected from a patient (e.g., via user device 120), and/or monitoring and/or therapy being used with a patient. For example, the therapy provider device 130 may configure the server 110, user device 120, and/or other compute devices (e.g., a caretaker device, supporter device, other provider device, etc.) to monitor certain information about a patient and/or provide certain content to a patient.

In some embodiments, information about a patient, e.g., collected by user device 120, therapy provider device 130, etc. can be provided to one or more other compute devices, e.g., server 110, compute device(s) 150, etc., which can be configured to process and/or analyze the information. For example, a data processing and/or machine learning device can be configured to receive raw information collected from or about a patient and process and/or analyze that information to derive other information about a patient (e.g., vocabulary, vocal-acoustic data, digital biomarker data, etc.). Further details of such data processing and/or analysis are described with reference to FIG. 2 below.

Compute device(s) 150 can include one or more additional compute devices, each including one or more processors and/or memories as described herein, that can be configured to perform certain functions. For example, compute device(s) 150 can include a data processing device, a machine learning device, a content creation or management device, etc. Further details of such devices are described with reference to FIG. 2. In some embodiments, compute device(s) 150 can include a supporter device, e.g., a device operated by a supporter (e.g., family, friend, caretaker, or other individual providing support and/or care to a patient). The support device can be configured to implement an application (e.g., a mobile application) that can assist in a patient's therapy. For example, the application can be configured to assist the supporter in learning more about a patient's conditions, providing encouragement to support the patient (e.g., recommend items to communicate and/or shared activities), etc. In some embodiments, the application can be configured to provide out-of-band information from the supporter to the system 100, such as, for example, information observed about the patient by the supporter. In some embodiments, the application can be configured to provide content that is linked to a patient's experience.

he compute devices described herein can communicate with one another via the network 102. The network 102 may be any type of network (e.g., a local area network (LAN), a wide area network (WAN), a virtual network, a telecommunications network) implemented as a wired network and/or wireless network and used to operatively couple the devices. As described in further detail herein, in some embodiments, for example, the system includes computers connected to each other via an Internet Service Provider (ISP) and the Internet. In some embodiments, a connection may be defined via the network between any two devices. As shown in FIG. 1, for example, a connection may be defined between one or more of server 110, user device 120 therapy provider device 130, database(s) 140, and compute device(s) 150.

In some embodiments, the compute devices may communicate with each other (e.g., send data to and/or receive data from) and with the network 102 via intermediate networks and/or alternate networks (not shown in FIG. 1). Such intermediate networks and/or alternate networks may be of a same type and/or a different type of network as network 102. Each compute device may be any type of device configured to send data over the network 102 to send and/or receive data from one or more of the other compute devices.

FIG. 2 depicts an example system 200, according to embodiments. The example system 200 can include compute devices and/or other components that are structurally and/or functionally similar to those of system 100. The system 200, similar to the system 100, can be configured to provide psychological education, psychological training tools and/or activities, psychological patient monitoring, coordinating care and psychological education with a patient's supporters (e.g., family members and/or caretakers), motivation, encouragement, appointment reminders, and the like.

The system 200 can include a connected infrastructure (e.g., server or sever-less cloud processing) of various compute devices. The compute devices can include, for example, a server 210, a mobile device 220, a content repository 242, a database 244, a raw data repository 246, a content creation tool 252, a machine learning system 254, and a data processing pipeline 256. In some embodiments, the system 200 can include a separate administration device (not depicted), e.g., implementing an administration tool (e.g., a website or desktop based program). In some embodiments, the system 200 can be managed via one or more of the server 210, mobile device 220, content creation tool 252, etc.

The server 210 can be structurally and/or functionally similar to server 110, described with reference to FIG. 1. For example, the server 210 can include a memory and a processor. The server 210 can be configured to perform one or more of: processing and/or analyzing data associated with a patient, evaluating a patient based on raw and/or processed data associated with the patient, generating and sending alerts to therapy providers, physicians, and/or caretakers regarding a patient, or determining content to provide to a patient before, during, and/or after receiving a treatment or therapy. In some embodiments, the server 210 can be configured to perform user authentication, process requests for retrieving or storing data relating to a patient's treatment, assign content for a patient and/or supporters (e.g., family, friends, and/or other caretakers), interpret questionnaire results, generate reports (e.g., PDF reports), schedule appointment for treatment and/or send reminders to patients and/or practitioners of appointments. The server 210 can be coupled to one or more databases, including, for example, a content repository 242, a database 244, and a raw data repository 246.

The mobile device 220 can be structurally and/or functionally similar to the user device 120, described with reference to FIG. 1. For example, the mobile device 220 can include a memory, a processor, a I/O device, a sensor, etc. In some embodiments, the mobile device 220 can be configured to implement a mobile application. The mobile application can be configured to present (e.g., display, present as audio) content that is assigned to a user and/or supporter. In some embodiments, content can be assigned to a user throughout a predefined period of time (e.g., a day, or throughout a course of treatment). Content can be presented for a predefined period of time, e.g., about 30 seconds to about 20 minutes, including all values and subranges in-between. Content can be delivered to a user, e.g., via mobile device 220, at periodic intervals, e.g., each day, each week, each month, etc. In some embodiments, the content delivered to a particular user can be based on rules or protocols assigned to different courses and/or assignments, as defined by the content creation tool 252 (described below).

In some embodiments, the mobile device 220 (e.g., via the mobile application) can track completion of activities including, for example, recording metrics of response time, activity choice, and responses provided by a user. In some embodiments, the mobile device 220 can record passive data including, for example, hand tremors, facial expressions, eye movement and pupillometry, and keyboard typing speed. In some embodiments, the mobile device 220 can be configured to send reward messages to users for completing an assignment or task associated with the content.

In some embodiments, content can involve interactions in group activities. For example, the mobile device 220 can present a virtual chat to a small group of patients that perform content and activities together. In some embodiments, the group activities can allow the group to participate and communicate in real-time or substantially real-time with each other and/or a therapist provider. In some embodiments, the group activities can allow the group to leave messages or complete activities for each other to be received or read by other group members at a later time period. In some embodiments, the mobile device 220 (e.g., via the mobile application) can be configured to receive and/or present push notifications, e.g., to remind users of upcoming assignments, appointments, group activities, therapy sessions, treatment sessions, etc. In some embodiments, the mobile device 220 (e.g., via the mobile application) can be configured to log a history of content, e.g., such that a user can review past content that they have consumed. In some embodiments, the mobile device 220 (e.g., via the mobile application) can provide an avatar creation function that allows users to choose and/or alter a virtual avatar. The virtual avatar can be used in group activities, guided journaling, dialogs, or other interactions in the mobile application.

In some embodiments, the system 200 can include external sensor(s) attached to a patient, e.g., biometric data from a wristband, ring, or other attached device. In some embodiments, the external sensors can be operatively coupled to a user device, such as, for example, the mobile device 220.

The content repository 242 can be configured to store content, e.g., for providing to a patient via mobile device 220 or another user device. Content can include passive information or interactive activities. Examples of content include: videos, articles including text and/or media, audio recordings, surveys or questionnaires including open-ended or close-ended questions, guided journaling activities or open-ended questions, meditation exercises, etc. In some embodiments, content can include dialog activities that allow a user to interact in a conversation or dialog with one or more virtual participants, where responses are pre-written options that lead users through different nodes in a dialog tree. A user can begin at one node in the dialog tree and move through that node depending on selections made by the user in response to the presented dialog. In some embodiments, content can include a series of open-ended questions that encourage or guide a user to a greater degree of understanding of a subject. In some embodiments, content can include meditation exercises with a voice and connected imagery to guide a user through breathing and/or thought exercises. In some embodiments, content can include one or more questions (e.g., questions in a questionnaire) that provoke one or more responses from a user, which can lead to haptic feedback. For example, as described in more detail with reference to FIGS. 9-16, a device (e.g., user device) can be configured to generate haptic feedback to interact with a patient, e.g., to communicate certain information relating to a user's response to the user.

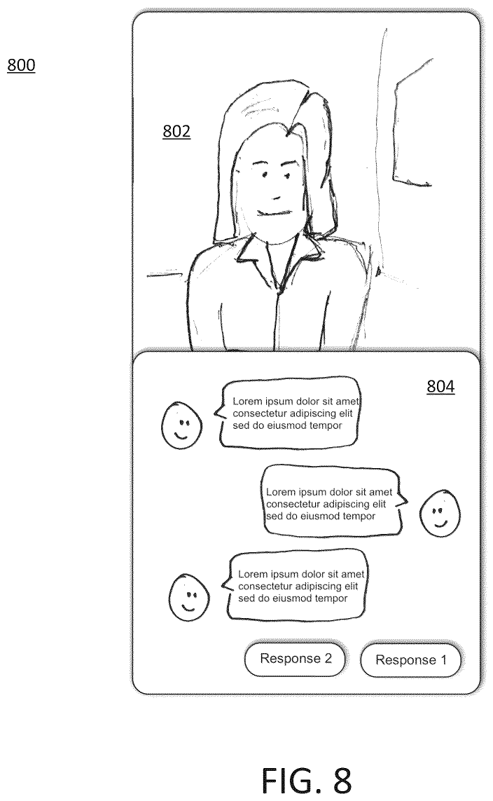

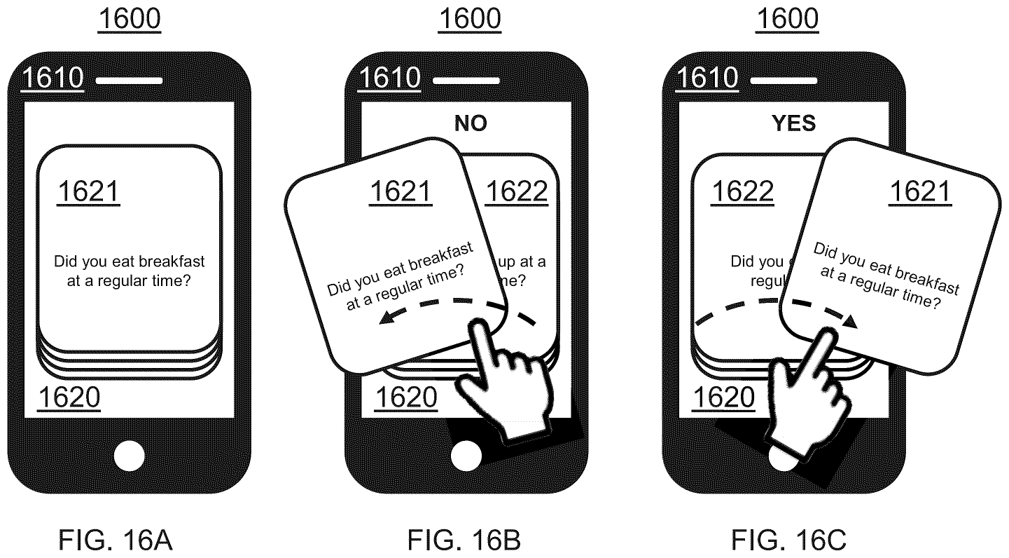

FIG. 8 depicts an example of a graphical user interface (GUI) 800 for delivering or presenting content to a user, e.g., on mobile device 220. The GUI 800 can include a first section 802 for presenting media, e.g., an image or video content. In some embodiments, the first section 802 can present a live or pre-recorded video feed of a therapy provider. The GUI 800 can also include a second section 804 for presenting a dialog, e.g., between a user and a therapy provider. In some embodiments, the user or the therapy provider can have an avatar or picture associated with that user or therapy provider, and that avatar or picture can be displayed alongside text inputted by the user or therapy provider in section 804. In some embodiments, the user and the therapy provider can have an open dialog. Alternatively or additionally, the user can be presented questions (e.g., a questionnaire) and asked to provide a response to those questions. For example, as depicted in FIG. 8, a therapy provider can ask the user a question and the user can be provided with two possible response options, i.e., “Response 1” and “Response 2,” as identified in selection buttons at a bottom of the GUI 800. In some embodiments, the user can be asked to respond by manipulating a slider bar or other user interface element. In some embodiments, the user can respond via gesture, such as swiping, as further discussed in context of FIGS. 16A, 16B, and 16C. In some embodiments, the user's response can cause the device to generate haptic feedback, e.g., similar to that described with reference to FIGS. 9-16. In some embodiments, the user can be asked to respond to a question vocally instead of by text or gesture. In some embodiments, the dialog can be used to infer a depression metric, concrete verses abstract thinking metric, or understanding of previously presented content, among other things.

While two sections are shown in the GUI 800, it can be appreciated that one or more additional sections can be provided in a GUI without departing from the scope of the present disclosure. For example, the GUI 800 can include additional sections providing media, questions (e.g., questionnaire), etc. In some embodiments, the GUI 800 can present pop-ups or sections that overlay other sections, e.g., to direct the user to specific content before viewing other content.

In some embodiments, content can be recursive, e.g., content can contain other content inline, and in some cases, certain content can block completion of its parent content until the content itself is completed. For example, a video can pause and a questionnaire can be presented on a screen, where the questionnaire must be completed before the video continues playing. In FIG. 8, for example, the dialog can be embedded in a video. As another example, an article can pause and cannot be read further (e.g., scrolled) until a video is watched. In some embodiments, the video also be recursive, for example, contain a questionnaire that must be completed before the video can resume and unlock the article for further reading.

Content can be analyzed and interpreted into metrics that are usable by other rules or triggers. For example, content can be analyzed and used to generate a metric indicative of a physiological state (e.g., depression), concrete versus abstract thinking, understanding of previously presented content, etc.

The content repository 242 can be operatively coupled to (e.g., via a network such as network 102) a content creation tool or application 252. The content creation tool 252 can be an application that is deployed on a compute device, such as, for example, a desktop or mobile application or a web-based application (e.g., executed on a server and accessed by a compute device). The content creation tool 252 can be used to create and/or edit content, organize content into courses and/or packages of information, schedule content for particular patients and/or groups of patients, set pre-requisite and/or predecessor content relationships, and/or the like.

In some embodiments, the system 200 can deliver content that can be used alongside (e.g., before, during or after) a therapeutic drug, device, or other treatment protocol (e.g., talk therapy). For example, the system 200 can be used with drug therapies including, for example, salvinorin A (sal A), ketamine or arketamine, 3,4-Methylenedioxymethamphetamine (MDMA), N-dimethyltryptamine (DMT), or ibogaine or noribogaine.

For example, during a pre-treatment phase, the system 200 can be configured to provide (e.g., via server 210 and/or user device 220, with information from content repository 242 and/or other components of the system 200) content to a user that prepares the user for a treatment and/or collect baseline patient data. In some embodiments, the system 200 can provide educational content (e.g., videos, articles, activities) for generic mindset and specific education of how a particular drug treatment can feel and/or affect a patient. In some embodiments, the system 200 can provide an introduction into behavioral activation content. In some embodiments, the system 200 can provide motivational interviewing and/or stories. In some embodiments, the system 200 can be configured to provide content that encourages and/or motivates a user to change.

In a post-treatment phase, the system 200 can be configured to provide content that assists a patient with processing and/or integrating his experience during the treatment. In some embodiments, the system 200 can provide psychoeducation skills content through articles, videos, interstitial questions (e.g., questionnaires), dialog trees (e.g., questionnaires), guided journaling, audio meditations, podcasts, etc. In some embodiments, the system 200 can provide motivational reminders and/or feedback from motivational interviewing. In some embodiments, the system 200 can provide group therapy activities. In some embodiments, the system 200 can provide questionnaires.

In some embodiments, the system 200 can be configured to assist a patient in long term management of a treatment outcome. For example, the system 200 can be configured to provide long-term monitoring via questionnaires, dialogs, digital biomarkers, etc. The system 200 can be configured to provide content for training a user on additional skills. The system 200 can be configured to provide group therapy activities with more advanced skills and/or subjects. The system 200 can be configured to provide digital pro re nata, e.g., by basing dosing and/or next treatment suggestions on content delivered to the user (e.g., coursework, assignments, referral to additional services, re-dosing with the original combination drug, etc.).

The raw data repository 246 can be configured to store information about a patient, e.g., collected via mobile device 220, sensor(s), and/or devices operated by other individuals that interact with the patient. Data collected by such devices can include, for example, timing data (e.g., time from a push notification to open, time to choose from available activities, hesitation time on questionnaires, gestures, reading speed, scroll distance, time from button down to button up), choice data (e.g., activities that are preferred or favorited, interpretation of questionnaire and interstitial question responses such as fantasy thinking, optimism/pessimism, and the like), phone movement data (e.g., number of steps during walking meditations, phone shake), and the like. Data collected by such devices can also include patient responses to interactive questionnaires, patient use and/or interpretation of text, vocal-acoustic data (e.g., voice tone, tonal range, vocal fry, inter-word pauses, diction and pronunciation), digital biomarker data (e.g., pupillometry, facial expressions, heart rate, etc.). Data collected by such devices can also include data collected from a patient during different activities, e.g., sleep, walking, during content delivery, etc.

The database 244 can be configured to store information for supporting the operation of the server 210, mobile device 220, and/or other components of system 200. In some embodiments, the database 244 can be configured to store processed patient data and/or analysis thereof, treatment and/or therapy protocols associated with patients and/or groups of patients, rules and/or metrics for evaluating patient data, historical data (e.g., patient data, therapy data, etc.), information regarding assignment of content to patients, machine learning models and/or algorithms, etc. In some embodiments, the database 244 can be coupled to a machine learning system 254, which can be configured to process and/or analyze raw patient data from raw data repository 246 and to provide such processed and/or analyzed data to the database 244 for storage.

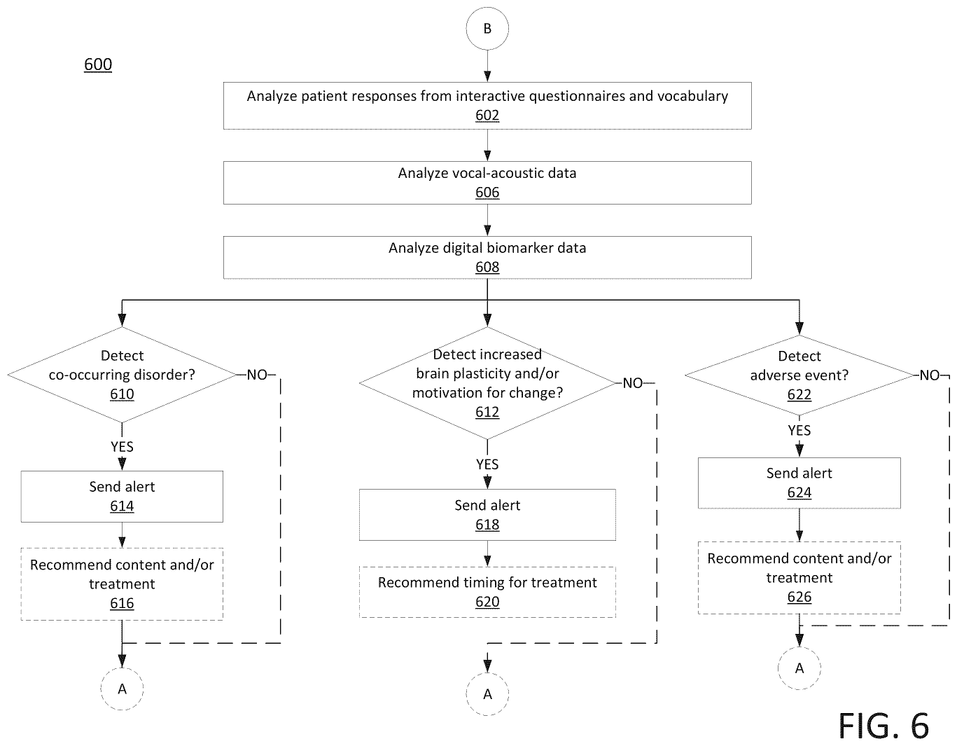

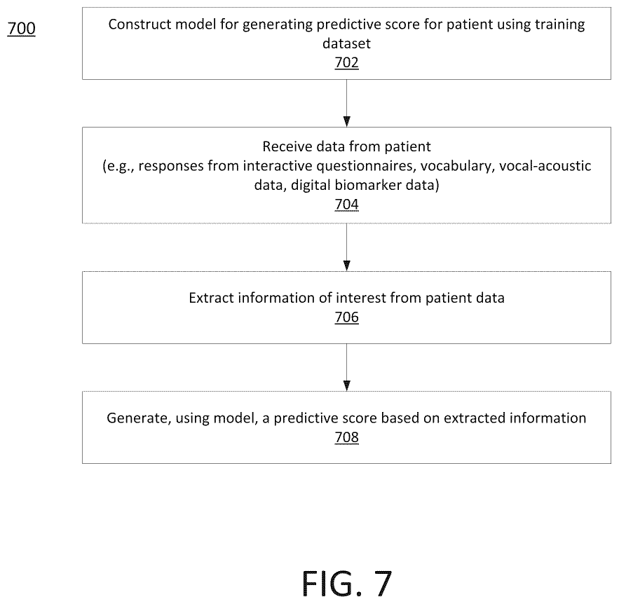

The machine learning system 254 can be configured to apply one or more machine learning models and/or algorithms (e.g., a rule-based model) to evaluate patient data. The machine learning system 254 can be operatively coupled to the raw data repository 246 and the database 244, and can extract relevant data from those to analyze. The machine learning system 254 can be implemented on one or more compute devices, and can include a memory and processor, such as those described with reference to the compute devices depicted in FIG. 1. In some embodiments, the machine learning system 254 can be configured to apply on or more of a general linear model, a neural network, a support vector machine (SVM), clustering, combinations thereof, and the like. In some embodiments, a machine learning model and/or algorithm can be used to process data initially collected from a patient to determine a baseline associated with the patient. Later data collected by the patient can be processed by the machine learning model and/or algorithm to generate a measure of a current state of the patient, and such can be compared to the baseline to evaluate the current state of the patient. Further details of such evaluation are described with reference to FIGS. 6 and 7.

The data processing pipeline 256 can be configured to process data received from the server 210, mobile device 220, or other components of the system 200. The data processing pipeline 256 can be implemented on one or more compute devices, and can include a memory and processor, such as those described with reference to the compute devices depicted in FIG. 1. In some embodiments, the data processing pipeline 256 can be configured to transport and/or process non-relational patient and provider data. In some embodiments, the data processing pipeline 256 can be configured to receive, process, and/or store (or send to the database 244 or the raw data repository 246 for storage) patient data including, for example, aural voice data, hand tremors, facial expressions, eye movement and/or pupillometry, keyboard typing speed, assignment completion timing, estimated reading speed, vocabulary use, etc.

1.2 Haptic Feedback

As described above, digital therapeutics can be used to assess and monitor patients' physical and mental health. For example, when a patient undergoes a drug treatment, the patient can use an electronic device such as a mobile device to provide health information for the medical health providers to assess and monitor the patient's health pre-treatment, during the treatment, and/or post-treatment, so that optimized/adjusted treatments can be given to the patient.

Questionnaires are known to be presented in a simple digital representation of paper questionnaires. Some known questionnaires add buttons or check boxes. These questionnaires, however, are one-way data transmission from the user of the mobile device to the device.

In some embodiments, embodiments described herein can combine haptic feedback into questionnaires to achieve two-way interactions and data transmission between the patient and the mobile device (and other compute devices in communication with the mobile device). In some embodiments, a set of questions can be given to a patient (or a user of a mobile device). When the patient provides input to the device to answer the questions, the device (or a mobile application on the device) can use haptic feedback (e.g., vibration) to interact with the patient. The vibration can be in different patterns in different situations.

In some implementations, for example during a psychoeducational session or delivery of digital content, a question, and a virtual interface element is presented to a user. The virtual interface element includes a plurality of selectable responses to the question. Each question is associated with a different measure of a parameter. The user selects a response from the plurality of selectable responses as a first input via the virtual interface element. A first haptic feedback is generated based on the first selectable response or the first input. When a user selects a second response from the plurality of selectable responses as a second input via the virtual interface element, where the second input represents a greater measure of the parameter than the first selectable response, a second haptic feedback is generated based on the second selectable response. The second haptic feedback has an intensity or frequency that is greater than the first haptic feedback. The first and second haptic feedback are different in waveform, intensity, or frequency.

For example, the mobile device (or the mobile application) can use the haptic feedback to alert the patients that their answer is straying from their last response (e.g., “how different do you feel today”). For another example, the device (or the mobile application) can use the haptic feedback to alert the patients that they are reaching an extreme (e.g., “this is the worst I've ever felt”). For another example, the device (or the mobile application) can use the haptic feedback to alert the patients on how their answer differs from the average or others in their group. In some embodiments, the haptic feedback for questions can be used with slider scales, increasing or decreasing haptic feedback as the patients move their finger. In some embodiments, haptic feedback for questions can be used in association or as feedback to user gestures. For example, a user may make a gesture to respond to a question. In response to the gesture, the mobile device may provide feedback indicating the speed of the user's response (e.g., a strong ‘yes’ or strong ‘no’ regarding a specific question).

In some embodiments, using the haptic feedback to interact with users of the mobile device or other electronic devices while they are answering questions can remind users of past responses or average responses to ground their current answer. In some examples, this can provide medical care providers, care takers, or other individuals more accurate responses.

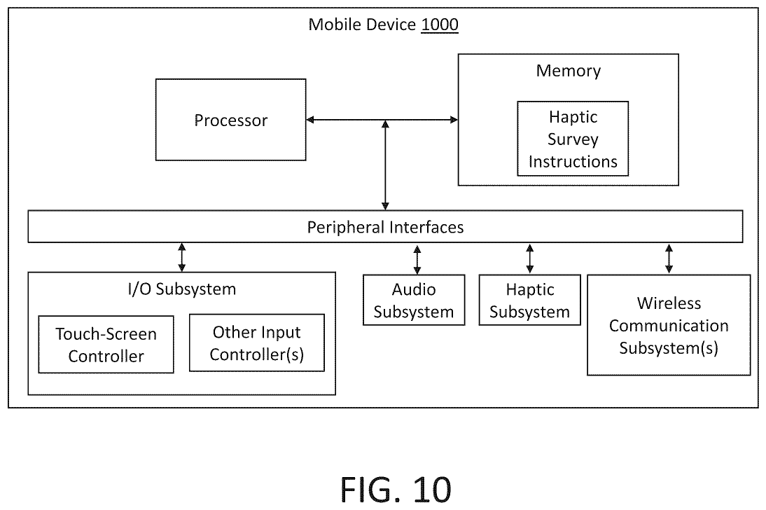

FIG. 9 illustrates an example schematic diagram illustrating a system 900 for implementing haptic feedback for questionnaires or a haptic questionnaire system 900, according to some embodiments. In some embodiments, the haptic questionnaire system 900 includes a first compute device such as a server 901 and a second compute device such as a user device 902 configured to communicate with the server 901 via a network 903. Alternatively, in some embodiments, the system 900 does not include a server 901 that communicates with a user device 902 but includes one or more compute devices such as user device(s) 902 having components that form an input/output (I/O) subsystem 923 (e.g., a display, keyboard, etc.) and a haptic feedback subsystem 924 (e.g., a vibration generating device such as, for example, a mechanical transducer, motor, speaker, etc.). Such an implementation is further described and illustrated with respect to FIG. 10.

The server 901 can be a compute device (or multiple compute devices) having a processor 911 and a memory 912 operatively coupled to the processor 911. In some instances, the server 901 can be any combination of hardware-based module (e.g., a field-programmable gate array (FPGA), an application specific integrated circuit (ASIC), a digital signal processor (DSP)) and/or software-based module (computer code stored in memory 912 and/or executed at the processor 911) capable of performing one or more specific functions associated with that module. In some instances, the server 901 can be a server such as, for example, a web server, an application server, a proxy server, a telnet server, a file transfer protocol (FTP) server, a mail server, a list server, a collaboration server and/or the like. In some instances, the server 901 can be a personal computing device such as a desktop computer, a laptop computer, a personal digital assistant (PDA), a standard mobile telephone, a tablet personal computer (PC), and/or so forth. In some embodiments, the capabilities provided by the server 901, as described herein, may be a deployment of a function on a serverless computing platform (or a web computing platform, or a cloud computing platform) such as, for example, AWS Lambda.

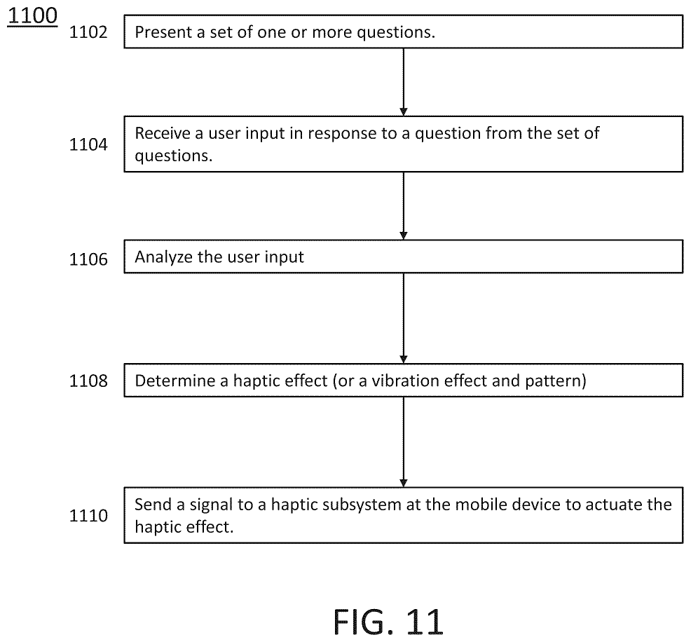

The memory 912 can be, for example, a random-access memory (RAM) (e.g., a dynamic RAM, a static RAM, etc.), a flash memory, a removable memory, a hard drive, a database and/or so forth. In some implementations, the memory 912 can include (or store), for example, a database, process, application, virtual machine, and/or other software modules (stored and/or executing in hardware) and/or hardware modules configured to execute a haptic questionnaire process as described with regards to FIG. 11. In such implementations, instructions for executing the haptic questionnaire process and/or the associated methods can be stored within the memory 912 and executed at the processor 911. In some implementations, the memory 912 can store questions (e.g., questionnaires), answers (e.g., responses to questionnaires), patient data, haptic questionnaire instructions, and/or the like. In some implementations, a database coupled to the server 901, the user device, 902, and/or a haptic feedback subsystem (not shown in FIG. 9) can store questions, answers, patient data, haptic questionnaire instructions, and/or the like.

The processor 911 can be configured to, for example, write data into and read data from the memory 912, and execute the instructions stored within the memory 912. The processor 911 can also be configured to execute and/or control, for example, the operations of other components of the server 901 (such as a network interface card, other peripheral processing components (not shown)). In some implementations, based on the instructions stored within the memory 912, the processor 911 can be configured to execute one or more steps of the haptic questionnaire process described with respect to FIG. 11.

The user device 902 can be a compute device having a processor 921 and a memory 922 operatively coupled to the processor 921. In some instances, the user device 902 can be a mobile device (e.g., a smartphone), a tablet personal computer, a personal computing device, a desktop computer a laptop computer, and/or the like. The user device 902 can include any combination of hardware-based module (e.g., a field-programmable gate array (FPGA), an application specific integrated circuit (ASIC), a digital signal processor (DSP)) and/or software-based module (computer code stored in memory 922 and/or executed at the processor 921) capable of performing one or more specific functions associated with that module.

The memory 922 can be, for example, a random-access memory (RAM) (e.g., a dynamic RAM, a static RAM, etc.), a flash memory, a removable memory, a hard drive, a database and/or so forth. In some implementations, the memory 922 can include (or store), for example, a database, process, application, virtual machine, and/or other software modules (stored and/or executing in hardware) and/or hardware modules configured to execute a haptic questionnaire process as described with regards to FIG. 11. In such implementations, instructions for executing the haptic questionnaire process and/or the associated methods can be stored within the memory 922 and executed at the processor 921. In some implementations, the memory 922 can store questions, answers, patient data, haptic questionnaire instructions, and/or the like.

The processor 921 can be configured to, for example, write data into and read data from the memory 922, and execute the instructions stored within the memory 922. The processor 921 can also be configured to execute and/or control, for example, the operations of other components of the user device 902 (such as a network interface card, other peripheral processing components (not shown), etc.). In some implementations, based on the instructions stored within the memory 922, the processor 921 can be configured to execute one or more steps of the haptic questionnaire process described herein (e.g., with respect to FIG. 11). In some implementations, the processor 921 and the processor 911 can be collectively configured to execute the haptic questionnaire process described herein (e.g., with respect to FIG. 11).

In some embodiments, the user device 902 can be an electronic device that is associated with a patient. In some embodiments, the user device 902 can be a mobile device (e.g., a smartphone, tablet, etc.), as further described with reference to FIG. 10. In some embodiments the user device may be a shared computer at a doctor's office, hospital or a treatment center.

In some embodiments, the user device 902 can be configured with a user interface, e.g., a graphical user interface, that presents one or more questions to a user. In some embodiments, the user device 902 can implement a mobile application that presents the user interface to a user. In some embodiments, the one or more questions can form a part of a questionnaire, e.g., for obtaining information about the user in relation to a drug treatment or therapy program. In some embodiments, the one or more questions can be provided during a digital therapy session, e.g., for treating a medical condition of a patient and/or preparing a patient for a drug treatment or therapy. In some embodiments, the one or more questions can be provided as part of a periodic questionnaire (e.g., a daily, weekly, or monthly check-in), whereby a patient is asked to provide information regarding a mental and/or physical state of the patient.

In some embodiments, the user device 902 can present one or more questions to a patient and transmit one or more responses from the patient to the server 901. The one or more questions and the one or more responses can have translations specific to the user's language layered with the questions and/or responses. For example, the user device 902 can present a question (e.g., “How are you feeling today?”) on a display or other user interface, and can receive an input (e.g., a touch input, gesture, microphone input, or keyboard entry) and transmit that input to the server 901 via network 903. In some embodiments, the inputs into the user device 902 can be transmitted in real time or substantially in real time (e.g., within about 1 to about 5 seconds) to the server 901. The server 901 can analyze the inputs from the user device 902 and determine whether to instruct the user device 902 to generate or produce some haptic effect (e.g., a vibration effect or pattern) based on the inputs. For example, the server 901 can have haptic questionnaire instructions stored that instruct the server 901 on how to analyze inputs and/or generate instructions to the user device 902 on what haptic effect to produce. In response to determining that a haptic effect should be provided at the user device 902, the server 901 can send one or more instructions back to the user device 902, e.g., instructing the user device to generate or produce a determined haptic effect (e.g., a vibration effect or pattern).

Alternatively or additionally, the user device 902 can present one or more questions to a patient and process or analyze one or more responses from the patient. For example, the user device 902 can present a question (e.g., “How are you feeling today?”) on a display or other user interface, and can receive an input (e.g., a touch input, gesture, microphone input, keyboard entry, etc.) after presenting the question. The user device 902 can have stored in memory (e.g., memory 922) one or more instructions (e.g., haptic questionnaire instructions) that instruct the user device 902 on how to process and/or analyze the input. For example, the user device 902 via processor 921 can be configured to process an input to provide a transformed or cleaned input. The user device 902 can pass the transformed or cleaned input to the server 901, and then wait to receive additional instructions from the server 901, e.g., for generating a haptic effect as described above. As another example, the user device 902 via processor 921 can be configured to analyze the input, for example, by comparing the input to a previous input provided by the user. The user device 902 can then determine whether to generate a haptic effect based on the comparison, as further described with respect to FIG. 11. In some embodiments, the user device 902 can have one or more questionnaire definition files stored, with each questionnaire definition file defining one or more questions, translations for prompting questions, rules for presenting questions on the user device, rules for presenting answers on the user device (for the user to input or select), associated inputs, and associated haptic feedback instructions. The questionnaire definition file can also include a function definition that converts a user input (i.e., answers to questions) into one or more haptic feedback. For example, each questionnaire definition file can define one or more haptic feedback or changes to one or more haptic feedback (e.g., a change in amplitude or intensity, or a change in type of haptic feedback pattern) based on one or more inputs received at the user device 902.

In some implementations, the system 900 for implementing haptic feedback for questionnaires or the haptic questionnaire system 900 can include a single device, such as the user device 902, having a processor 921, a memory 922, an input/output (I/O) subsystem 923 (including, for example, a display and/or one or more input devices), and a haptic feedback subsystem 924 (e.g., a motor or other peripheral device) capable of providing haptic feedback. For example, the system 900 can be implemented as a mobile device (having a mobile application executed by the processor of the mobile device). In some implementations, the system 900 can include multiple devices, e.g., one or more user device(s) 902. A first device can include, for example, a processor 921, a memory 922, and a display (e.g., a liquid-crystal display (LCD), a Cathode Ray Tube (CRT) display, a touchscreen display, etc.) and an input device (e.g., a keyboard) that form part of an I/O subsystem 923, and a second device can include a haptic feedback subsystem 924 that is in communication with the first device (e.g., a speaker embedded in a seat or other environment around a user). For example, the user can provide answers to the questions via the first device and receive haptic feedback via the second device. In some implementations, the first device can be configured to be in communication with the server 901 and the second device can be configured to be in communication with the first device. In some implementations, the first device and the second device can be configured to be in communication with the server 901. In some implementations, a database coupled to the server 901, the user device, 902, or the haptic feedback subsystem (not shown in FIG. 9) can store questionnaire questions, questionnaire answers, patient data, haptic questionnaire instructions, and/or the like.

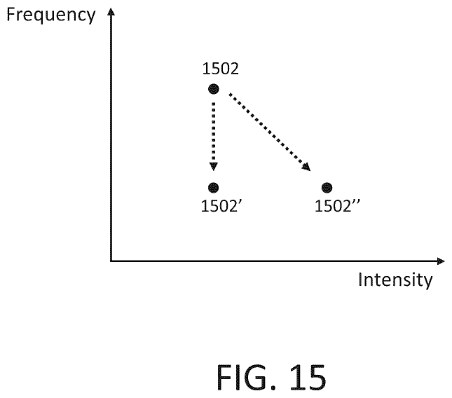

Examples of haptic effects include a vibration having different characteristics on a user device 902. The intensity, duration, pattern, and/or other characteristics of each haptic effect can vary. For example, a haptic effect can be associated with n number of characteristics that can each be varied. FIG. 15 depicts an example where a haptic effect is associated with two characteristics (e.g., intensity and frequency), and each can be varied along an axis. The haptic effect at any point in time can be represented by a point 1502 in the coordinate space. For example, in response to a user positioning a slider bar at a first position, the haptic effect can be represented by point 1502. When the user moves the slider bar to a second position, the haptic effect can change in frequency, e.g., to point 1502′, or in both frequency and intensity, e.g., to point 1502″. Other combinations of changes, e.g., only a change in intensity, an increase in intensity and/or frequency, etc. can also be implemented based on an input from the user. To further expand on the model described with reference to FIG. 15, it can be appreciated that a haptic effect can be associated with any number of characteristics, and that each characteristic can be adjusted along one or more axes, such that a haptic effect can be associated with n number of axes. In some implementations, for example, three axes representing intensity, frequency and pattern of the haptic feedback can be used. In such implementations, depending on the input by the user, one or more of intensity, frequency and pattern of the haptic feedback can change. Changes in the one or more characteristics can be used to indicate different information to a user (e.g., amount of time that user is taking to respond to a question, how response compares to baseline or historical responses, etc.).

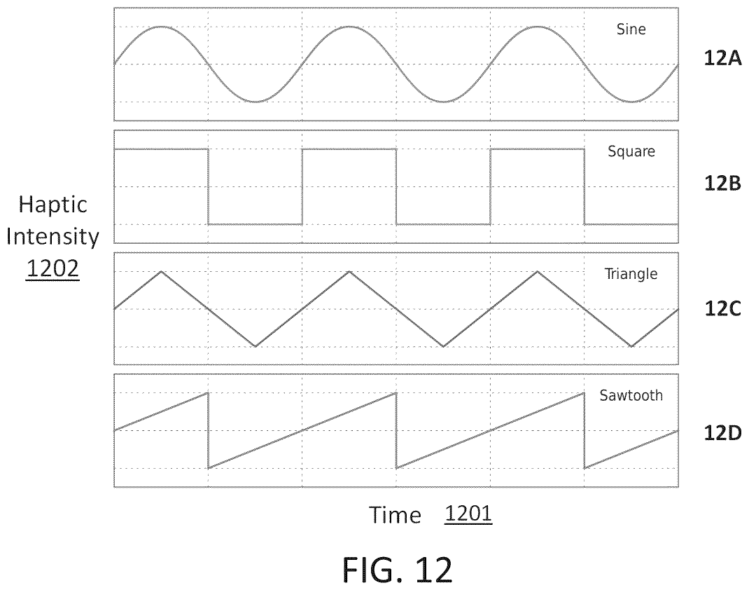

In some embodiments, the haptic effect can be associated with a particular type of pattern. FIG. 12 shows examples of haptic effect patterns, according to some embodiments. In some implementations, the intensity of the vibration 1202 can change as a function of time 1201, in a sine wave (12A), a square wave (12B), a triangle wave (12C), a sawtooth wave (12D), a combination of any of the above vibrating patterns, and/or the like. In some implementations, the haptic effect can be pulses of vibration having a pre-determined or adjustable frequency, amplitude, etc. For example, the vibration pulses can have a pattern of vibrating at a first intensity every five seconds, or a gradual pulse (e.g., a first vibration intensity pulsed every three seconds for the first 10 seconds and then change to a second vibration intensity pulsed at every two seconds for 15 seconds). For example, when the user device 902 presents a question (e.g., “How are you feeling today?”) on a display or other user interface, the user device can receive an input from the patient indicating her status today. When the patient's answer differs from the patient's answer from yesterday, the user device can generate a pulsed vibration as a haptic feedback, informing the patient that the answer is different from yesterday. The user device 902 can increase the intensity of the vibration, increase the frequency of the vibration, change a pattern of the vibration, or change another characteristic of the vibration when the deviation between the patient's answer today and the patient's answer yesterday increases. In some embodiments, the haptic effect can have a predefined attack and/or decay pattern. For example, the haptic effect can have an attack pattern and/or decay pattern that is defined by a function (e.g., an easing function).

Returning to FIG. 9, in some implementations, the patient's input to the user device 902 (to answer questionnaire questions) can be continuous (e.g., through a sliding scale) or discrete (e.g., multiple choice questions). The user device 902 (or in some implementations, the server 901) can generate haptic effect based on the continuous input and the discrete input. When the user device 902 receives discrete inputs from the user, the user device 902 can generate haptic effect based on the discrete input itself, and/or other user reactions to the questionnaire questions (e.g., user's hover or hesitation state).

In some embodiments, examples of haptic effects can include with sound (e.g., tone, volume or specific audio files), visual (e.g., pop-up windows on the user interface, floating windows), a text message, and/or the like. In some embodiments, the user device can generate combinations of different types of haptic effects (e.g., vibration and sound).

FIG. 10 illustrates an example schematic diagram illustrating a mobile device 1000 including a haptic subsystem, according to some embodiments. In some embodiments, the mobile device 1000 is physically and/or functionally similar to the user device 902 discussed with regards to FIG. 9. In some embodiments, the mobile device 1000 can be configured to be communicating with the server 901 via the network 903 to execute the haptic questionnaire process described with respect to FIG. 11. In some embodiments, the mobile device 1000 does not need to communicate with a server and the mobile device 1000 itself can be configured to execute the haptic questionnaire process described with respect to FIG. 11. In some embodiments, the mobile device 1000 includes one or more of a processor, a memory, peripheral interfaces, a input/output (I/O) subsystem, an audio subsystem, a haptic subsystem, a wireless communication subsystem, a camera subsystem, and/or the like. The various components in mobile device 1000, for example, can be coupled by one or more communication buses or signal lines. Sensors, devices, and subsystems can be coupled to peripheral interfaces to facilitate multiple functionalities. Communication functions can be facilitated through one or more wireless communication subsystems, which can include receivers and/or transmitters, such as, for example, radiofrequency and/or optical (e.g., infrared) receivers and transmitters. The audio subsystem can be coupled to a speaker and a microphone to facilitate voice-enabled functions, such as voice recognition, voice replication, digital recording, and telephony functions. I/O subsystem can include touch-screen controller and/or other input controller(s). Touch-screen controller can be coupled to a touch-screen or pad. Touch-screen and touch-screen controller can, for example, detect contact and movement using any of a plurality of touch sensitivity technologies.

The haptic subsystem can be utilized to facilitate haptic feedback, such as vibration, force, and/or motions. The haptic subsystem can include, for example, a spinning motor (e.g., an eccentric rotating mass or ERM), a servo motor, a piezoelectric motor, a speaker, a magnetic actuator (thumper), a taptic engine (a linear resonant actuator; or Apple's taptic engine), a Piezoelectric actuator, and/or the like.

The memory of the mobile device 1000 can be, for example, a random-access memory (RAM) (e.g., a dynamic RAM, a static RAM), a flash memory, a removable memory, a hard drive, a database and/or so forth. In some implementations, the memory can include (or store), for example, a database, process, application, virtual machine, and/or other software modules (stored and/or executing in hardware) and/or hardware modules configured to execute a haptic questionnaire process as described with regards to FIG. 11. In such implementations, instructions for executing the haptic questionnaire process and/or the associated methods can be stored within the memory and executed at the processor. In some implementations, the memory can store questionnaire questions, questionnaire answers, patient data, haptic questionnaire instructions, haptic questionnaire function definitions, and/or the like.

The memory can include haptic questionnaire instructions or function definitions. Haptic instructions can be configured to cause the mobile device 1000 to perform haptic-based operations, for example providing haptic feedback to a user of the mobile device 1000 as described in reference to FIG. 11.

The processor of the mobile device 1000 can be configured to, for example, write data into and read data from the memory, and execute the instructions stored within the memory. The processor can also be configured to execute and/or control, for example, the operations of other components of the mobile device. In some implementations, based on the instructions stored within the memory, the processor can be configured to execute the haptic questionnaire process described with respect to FIG. 11.

FIG. 11 illustrates a flow chart of an example haptic questionnaire process, according to some embodiments. This haptic questionnaire process 1100 can be implemented at a processor and/or a memory (e.g., processor 911 or memory 912 at the server 901 as discussed with respect to FIG. 9, the processor 921 or memory 922 at the user device 902 as described with respect to FIG. 9, and/or the processor or memory at the mobile device 1000 discussed with respect to FIG. 10).

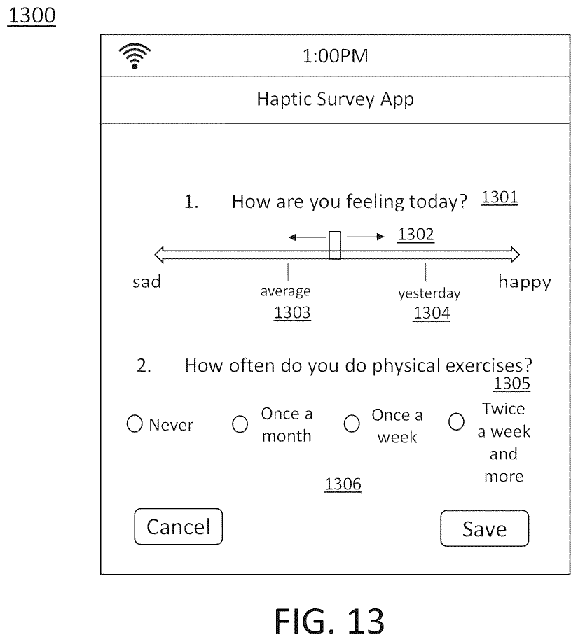

At step 1102, the haptic questionnaire process includes presenting a set of questionnaire questions, e.g., on a user interface of a user device (e.g., user device 902 or mobile device 1000). FIG. 13 shows an example user interface 1300 of the user device, according to some embodiments. In an embodiment, a questionnaire question 1301 can be “how are you feeling today?” The processor can present a slide bar 1302 from “sad” to “happy”. The user can tap and move the slide bar to indicate a mood between these two end points. In some implementations, the slide bar can show a line indicating the user's answer entered yesterday 1304, and/or a line indicating the user's average answer to the question 1303. As the user moves the slide bar 1302 away from the line 1303 or 1304, the user device generates a haptic effect to provide feedback to the user on the difference between their previous answers (e.g., yesterday's answer or the average answer) and their current answer. The feedback can help anchor the user to yesterday's answer or the average answer. The effect in this example is to mimic a therapist asking “are you sure you feel that much better? That's a lot”. This type of feedback can help patients with indications such as bi-polar disorders that may cause the patient to have large, quick swings in mood.

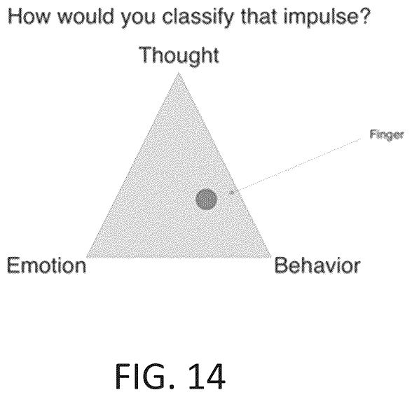

For another example, a questionnaire question 1305 can be “how often do you do physical exercises?” The processor can present multiple choices (or discrete inputs) 1306 for the user to choose the closet answer. The haptic questionnaire process can provide different types of answer choices, including, but are not limited to, a Visual Acuity Scale (e.g., a slide bar 1302), discrete inputs (or multiple choices 1306), a grid input (having two dimensions: a horizontal dimension and a vertical dimension with each dimension being used as an input to be provided to the haptic function) and/or the like. In some embodiments, the haptic questionnaire process can provide an answer format in multiple axes (or dimensions) displayed, for example, as a geometric shape in which the user can move their finger (or tap on the screen of the user device) to indicate the interplay between multiple choices. FIG. 14 is an example answer format having multiple axes, according to some embodiments. For example, the questionnaire question can be “how would you classify that impulse?” The answer can relate to three categories including behavior, emotion, and thought. The user can tap on the screen and move the finger to classify the impulse based on the categories of behavior, emotion, and thought. FIGS. 16A, 16B, and 16C, described below, also shows examples of a graphical interface 1600 through which a user interacts. Such graphical interface can also be used in conjunction with step 1102.

At step 1104, the haptic questionnaire process includes receiving a user input in response to a questionnaire question from the set of questionnaire questions, for example, through user interfaces shown in FIGS. 13, 14, 16A, 16B, and 16C.

At step 1106, the haptic questionnaire process includes analyzing the user input. For example, the processor can analyze the user input in comparison to a previous user input or a baseline in response to the questionnaire question, e.g., by measuring or assessing a difference between the user input and the previous user input or baseline (e.g., determining whether the user input differs from the previous user input or baseline by a predetermined amount or percentage). The processor can then generate a comparison result based on the analysis.

At step 1108, the haptic questionnaire process includes determining whether to provide a haptic effect (e.g., a vibration effect or pattern). For example, the processor can determine to provide a haptic effect when a comparison result between a user input and a previous user input or baseline meets certain criteria (e.g., when the comparison result reaches a certain threshold value, etc.). As another example, the processor can be configured to provide a haptic effect that increases in intensity or frequency as a user's response to a question increases relative to a baseline or predetermined measure (e.g., as a user moves a slider scale).

At step 1110, the haptic questionnaire process includes sending a signal to a haptic subsystem at the mobile device to actuate the haptic effect. In some embodiments, the processor can be the processor of a server (e.g., processor 911 of the server 901), and can be configured to analyze the user input and send an instruction to a user device (e.g., user device 902, mobile device 1000) to cause the user device to send the signal to the haptic subsystem for actuating the haptic effect. In some embodiments, an onboard processor of a user device (e.g., processor of the mobile device 1000) can be configured to analyze the user input and send the signal to the haptic subsystem for actuating the haptic effect.

While examples and methods described herein relate one or more haptic effects to questionnaires and/or questions contained in questionnaires, it can be appreciated that any one of the haptic feedback systems and/or components described herein can be used in other settings, e.g., to provide feedback while a user is adjusting settings (e.g., on a mobile device or tablet, such as in a vehicle), to provide feedback in response to questions that are not included in a questionnaire, to provide feedback while a user is engaging in certain activity (e.g., workouts, exercises, etc.), etc. Haptic effects as described herein can be varied accordingly to provide feedback in such settings.

FIGS. 16A, 16B, and 16C illustrate a user interface 1600 that is presented on a device 1610 (e.g., similar to user device 120), where a user can use gestures in response to questions in a questionnaire. FIG. 16A shows the user interface 1600 with a touchscreen display 1620 (e.g., a type of interactive display) displaying a graphical object 1621. The graphical object 1621 may be in a stack or collection of other objects, as shown. The collection of objects may be analogous to a stack of cards or paper. The graphical object 1621 includes a question—in this case, “Did you eat breakfast at a regular time?” As shown in FIG. 16B or 16C, the user interacts with graphical object 1621 by making a gesture with the user's hand. The type of gesture may correspond to a particular answer to a question. As shown in FIG. 16B, the user's hand “swipes” left, and that corresponds to a “NO” response. As shown in FIG. 16C, the user's hand “swipes” right, and that corresponds to a “YES” response. Once the user answers the question on graphical object 1621, a new graphical object 1622 may be revealed. Graphical object 1622 may display another question—in this case, “Did you get up at a regular time?” The user may again interact with the device 1610 by gesturing to provide responses to the question displayed on graphical object 1622.

The graphical objects 1621 and 1622 may be displayed on the touchscreen display 1620 in sequence, or may be displayed simultaneously. The user may be able to interact to respond to the questions on the graphical objects only one at a time, or the user may be able to interact with multiple graphical objects simultaneously. For example, a user could select multiple graphical objects and then use a single gesture to respond to the collection. The content of questions presented on graphical objects may be invariable, or the content may vary based on answers to previous questions. Gesturing may be the only mode for interacting with the display when responding to the questionnaire, or other modes of interaction may be simultaneously available (e.g., tapping, sliding a slider, keyboard input, voice input, etc.).

Questions may be binary (e.g., YES/NO, TRUE/FALSE) or may have more than two possible answers. In the latter case, three or more gestures may be possible inputs. As another option, the degree of a given gesture may provide additional information. For example, if a question requests the user to provide an answer within a range (e.g., question 1301 in FIG. 13), the intensity of a gesture may be sensed. A more intense gesture (e.g., a faster or quicker gesture) may indicate a larger number or degree than a less intense gesture. Similarly, a spatially longer gesture may indicate a larger number or degree than a spatially shorter gesture.

Gestures may be sensed through a touchscreen, as on touchscreen display 1620. Gestures may be sensed optically via a camera or with an infrared sensing system, located either on the device 1610 or externally. Gestures may be reconfigurable or assignable to different types of answers. For example, a swipe-right gesture could be assigned as “YES” or as “NO.” Further as an example, a swipe-up gesture could be reassigned to either “YES” or “NO” per the user's or administrator's preferences.

Gestures may be swipe(s), tap-and-release, or tap-and-hold, or combinations thereof. Gestures may include a directional component, including swipe-right, swipe-left, swipe-up, swipe-down, or swipe-diagonally (at various angles), or combinations thereof. The start location and/or end location may correspond to different gestures. Gesture(s) that start and/or end in different locations than other gesture(s) may indicate different answers to the questions. Different gestures may be sensed based on the user using a different number of fingers (e.g., one finger, two finger, etc.). Gestures may correspond to a number of taps (e.g., one tap, two taps, etc.) and optionally by a number of fingers making the taps. Gestures may correspond to multiple fingers in multiple locations moving in different directions (e.g., pinch, un-pinch, twist, etc.). A given gesture may be a combination of the aforementioned behaviors. For example, a gesture may be touch-and-hold at a specific start location (e.g., graphical object 1621) with two fingers and then swipe-right while still holding the two fingers to the touchscreen display. As mentioned above, the intensity of a gesture may provide additional information or different intensities may correspond to different gestures. Similarly, a spatially longer gesture may indicate a larger number or degree than a spatially shorter gesture. Device 1610 or another device could be used to record and assign custom gestures by recording and analyzing the input given by the user making such a gesture (through an appropriate sensing mode, such as touchscreen display 1620 or camera).

According to some embodiments, a user's gesture could be further analyzed to gather information about the user. For example, machine learning or AI algorithms could measure and classify one or more qualities of a given gesture or set of gestures. For example, response time and speed of gesture could indicate confidence in the user's answer, speed of gesture could be an indication of mental state/mood/energy, speed of response can be used to establish and account for the user's cognitive load signature (how quickly they can think/answer, in general). Such assessment and/or classification could be performed by one or more processing systems described herein.

2. Methods

2.1 Patient Data Collection and Analysis

FIG. 3 is a data flow diagram illustrating information exchanged and collected between different components of a system 300, according to embodiments described herein. The components of the system 300 can be structurally and/or functionally similar to those described above with reference to systems 100 and 200 depicted in FIGS. 1 and 2, respectively. As depicted in FIG. 3, a server 310 can be configured to process assignments, e.g., including various content as described above, for a patient. In an embodiment, the server 310 can send a push notification for an assignment to a mobile device 320 associated with the patient. The push notification can include or direct the patient to, e.g., via a mobile application on the mobile device 320, one or more questions associated with the assignment. The patient can provide responses to the one or more questions at the mobile device 320, which can then be provided back to the server 310. The server 310 can send the responses to a data processing pipeline 356, which can process the responses.

Additionally or alternatively, the server 310 can also receive other information associated with the completion of the assignment and evaluate that information (e.g., by calculating assignment interpretations), and send such information and/or its evaluation of the information onto the data processing pipeline 356. Additionally or alternatively, the mobile device 320 can send timing metrics (e.g., timing associated with completion of assignment and/or answering specific questions) to the data processing pipeline 356. The data processing pipeline 356, after processing the data received, can send that information to a raw data repository 346 or some other database for storage.

2.2 Patient Onboarding

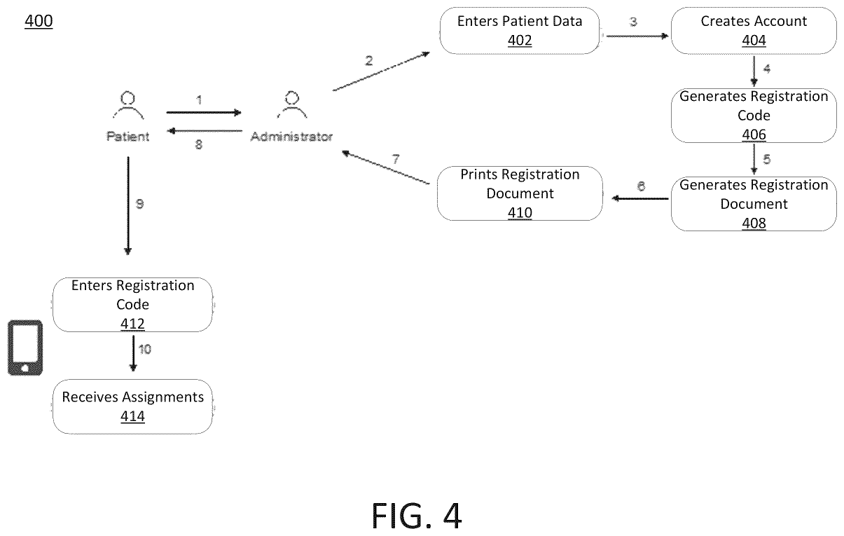

FIG. 4 depicts a flow diagram 400 for onboarding a new patient into a system, according to embodiments described herein. As depicted, a patient can interact with an administrator, e.g., via a user device (e.g., user device 120 or mobile device 220), and the administrator can enter patient data into a database, at 402. The patient data can be used to create an account for the user, at 404. For example, a server (e.g., server 110, 210) can create an account for the user using the patient data. A registration code can be generated, e.g., via the server, at 406. And a registration document including the registration code can be generated, e.g., via the server, at 408. The registration document can be printed, at 410, and provided to the administrator for providing to the patient. The patient can use the registration code in the registration document to register for a digital therapy course, at 412. For example, the patient can enter the registration code into a mobile application for providing the digital therapy course, as described herein. The user can then receive assignments (e.g., content) at the user device, at 414.

In some embodiments, systems and devices described herein can be configured to generate a unique registration code at 406 that indicates the particular course and/or assignment(s) that should be delivered to a patient, e.g., based on patient data entered at 402. For example, depending on the particular treatment and/or therapy desired and/or suitable for the patient, systems and devices described herein can be configured to generate a registration code that, upon being entered by the patient into the user device, can cause the user device to present particular assignments to the patient. The assignments can be selected to provide specific educational content and/or psychological activities to the patient based on the patient data.

2.3 Digital Therapy

Traditional talk therapy can be scheduled between a patient and a practitioner, during a mutually available time. Due to the overhead of travel, office scheduling and staff, and other reasons, these meetings can be usually scheduled in larger blocks of time, such as an hour or more. Patients in many mental health indications may not have the attention span for these long meetings, and many not have the ability to schedule meetings during typical working hours.

Assigning therapeutic content via a patient device (e.g., a mobile device) allows patients to receive smaller and manageable sessions of information, on a more frequent basis, and/or at a time that is more workable for their schedule. Information can be delivered according to a spaced periodic schedule, which can increase retention of the information.

In some embodiments, information can be provided in a collection of assignments that are assigned based on a manifest or schedule. The manifest or schedule can be set by a therapy provider and/or set according to certain predefined algorithms based on patient data. The content that is assigned may be a combination of content types as described above.

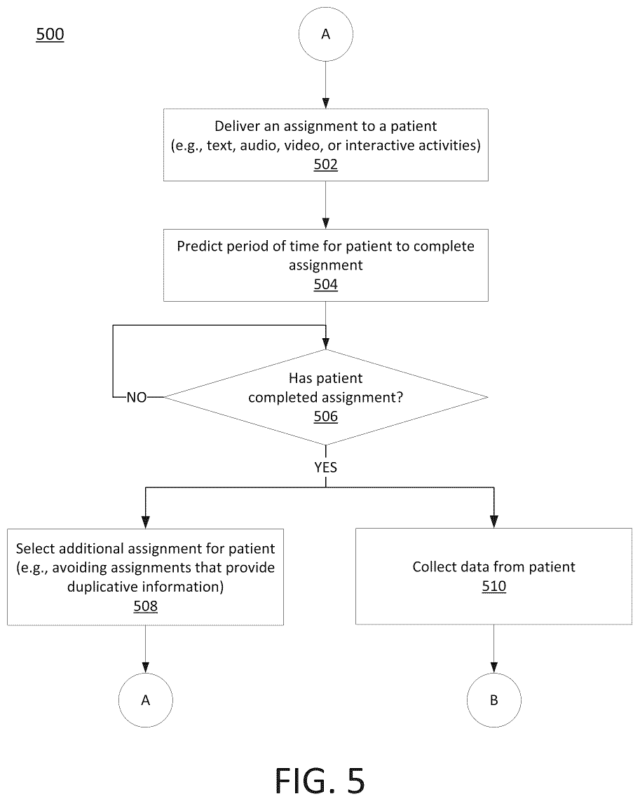

FIG. 5 is a flow chart illustrating a method 500 of delivering content to a patient, according to embodiments described herein. The content can be delivered to the patient for education, data-gathering, team-building, and/or entertainment. This method 500 can be implemented at a processor and/or a memory (e.g., processor 112 or memory 114 at the server 110 as discussed with respect to FIG. 1, the processor 122 or memory 124 at the user device 120 as described with respect to FIG. 1, the processor or memory at the server 210 and/or the mobile device 220 discussed with respect to FIG. 2, and/or the processor or memory at the server 310 and/or the mobile device 320 discussed with respect to FIG. 3).

At 502, an assignment including certain content (e.g., text, audio, video, or interactive activities) can be delivered to a patient. The assignment can be delivered, for example, via a mobile application implemented on a user device (e.g., user device 120, mobile device 220, mobile device 320). The assignment can include educational content relating to an indication of the patient, a drug that the patient may receive or have received, and/or any co-occurring disorders that may present themselves to a therapist, doctor, or the system. In some embodiments, the assignments can be delivered as push notifications on a mobile application running on the user device. The assignments can be delivered on a periodic basis, e.g., at multiple times during a day, week, month, etc.

In some embodiments, the delivery of an assignment can be timed such that it does not overwhelm a user by giving them too many assignments within a predefined interval. At 504, a period of time for the patient to complete the assignment can be predicted. The period of time for completing the assignment can be predicted, for example, by a server (e.g., server 110, 210, 310) or the user device, e.g., based on historical data associated with the patient. In some embodiments, an algorithm can be used to predict the period of time for the patient to complete the assignment, where the algorithm receives as inputs attributes of the assigned content (e.g., length, number of interstitial interactive questions, complexity of vocabulary, complexity of activities and/or tasks, etc.) and the patient's historical completion rates and metrics (e.g., number of assignments completed per day or other time period, calculated reading speed, calculated attention span).

At 506, the mobile device, server, or other component of systems described herein can determine whether the patient has completed the assignment and, optionally, can log the time for completion for further analysis or evaluation of the patient. In some embodiments, in response to determining that the patient has completed the assignment, the mobile device, server, or other component of systems described herein can select an additional assignment for the patient. Since assignments from different courses of treatment can be duplicative, or different assignments can provide substantially identical information to a therapist or other healthcare professional, systems and devices described herein can be configured to select assignments that are not duplicative (e.g., remove or skip assignments). The method 500 can then return to 502, where the subsequent assignment is delivered to the patient. In some embodiments, the mobile device server, or other component of systems described herein can collect data from the patient, at 510. Such components can collect the patient data during or after completion of the assignment. The collected data can be provided to other components of systems described herein, such as the server, data processing pipeline, machine learning system, etc. for further processing and/or analysis.

FIG. 6 depicts a flow chart of a method 600 for processing and/or analyzing patient data. This method 600 can be implemented at a processor and/or a memory (e.g., processor 112 or memory 114 at the server 110 as discussed with respect to FIG. 1, the processor 122 or memory 124 at the user device 120 as described with respect to FIG. 1, the processor or memory at the server 210, the mobile device 220, the data processing pipeline 256, the machine learning system 254, and/or other compute devices discussed with respect to FIG. 2, and/or the processor or memory at the server 310, the mobile device 320, and/or the data processing pipeline 356 discussed with respect to FIG. 3).

As depicted in FIG. 6, systems and devices described herein can be configured to analyze one or more of patient responses from interactive questionnaires and questionnaires and/or vocabulary from patient responses, at 602, vocal-acoustic data (e.g., voice tone, tonal range, vocal fry, inter-word pauses, diction and pronunciation), at 606, or digital biomarker data (e.g., decision hesitation time, activity choice, pupillometry and facial expressions), at 608, as well as any other data that can be collected from a patient via compute device(s) and sensor(s) described herein.

In some embodiments, systems and devices can be configured to detect or predict co-occurring disorders, e.g., to depression, PTSD, substance use disorder, etc. based on the analysis of the patient data, at 610. In some embodiments, co-occurring disorders can be detected via explicit questions in questionnaires (e.g., “How much did you sleep last night?”), passive monitoring (e.g., how much did a wearable device or other sensor detect that a user has slept last night), or indirect questioning in content, dialogs, and/or group activities (e.g., a user mentioning tiredness on several occasions). In response to detecting a co-occurring disorder, systems and devices can be configured to generate and send an alert to a physician and/or therapist, at 614, and/or recommend content or treatment based on such detection, at 616. For example, systems and devices can be configured to recommend a change in content (e.g., a different series of assignments or a different type of content) to present to the patient, or recommend certain treatment or therapy for the patient (e.g., dosing strategy, timing for dosing and/or other therapeutic activities such as talk therapy, medication, check-ups, etc.), based on the analysis of the patient data. If no co-occurring disorder is detected, systems and devices can continue to provide additional assignments to the patient and/or terminate the digital therapy.

In some embodiments, systems and devices can be configured to detect that a patient is in a suitable mindset for receiving a drug, therapy, etc. In some embodiments, systems and devices can detect an increased brain plasticity and/or motivation for change using explicit questioning, passive monitoring, and/or indirect questioning. For example, systems and devices can detect an increased brain plasticity and/or motivation for change based on the analysis of the patient data, at 612. In some implementations, systems and methods described herein can use software model(s) to generate a predictive score indictive of a state of the subject. The software model(s) can be, for example, an artificial intelligence (AI) model(s), a machine learning (ML) model(s), an analytical model(s), a rule based model(s), or a mathematical model(s). For example, systems and methods described herein can use a machine learning model or algorithm trained to generate a score indictive of a state of the subject. In some implementations, machine learning model(s) can include: a general linear model, a neural network, a support vector machine (SVM), clustering, or combinations thereof. The machine learning model(s) can be constructed and trained using a training dataset, e.g., using supervised learning, unsupervised learning, or reinforcement learning. The training data set can include a historical dataset from the subject. The historical dataset can include: historical biological data of the subject, historical digital biomarker data of the subject, and historical responses to questions associated with digital content by the subject. The historical biological data of the subject include at least one of: historical heart beat data, historical heart rate data, historical blood pressure data, historical body temperature, historical vocal-acoustic data, or historical electrocardiogram data. The historical digital biomarker data of the subject includes at least one of: historical activity data, historical psychomotor data, historical response time data of responses to questions associated with the digital content, historical facial expression data, historical pupillometry, or historical hand gesture data. The historical responses to the questions associated with the digital content by the subject include at least one of: historical self-reported activity data, historical self-reported condition data, or historical patient responses to questionnaires.

After the machine learning model(s) is trained using the training data, the systems and methods described in FIG. 6 steps 602, 604, 608, and 612 can be implemented using the trained machine learning model(s). For example, a set of psychoeducational sessions including digital content is provided to the subject. A set of data streams associated with the subject can be collected and using the trained machine learning model(s), a predictive score indictive of a state of the subject can be generated. A set of data streams associated with the subject while providing the set of psychoeducational sessions is collected. The set of data streams can include at least one of: biological data of the subject, digital biomarker data of the subject, or responses to questions associated with the digital content by the subject. The biological data of the subject include at least one of: heart beat data, heart rate data, blood pressure data, body temperature, vocal-acoustic data, or electrocardiogram data. The digital biomarker data of the subject includes at least one of: activity data, psychomotor data, response time data of responses to questions associated with the digital content, facial expression data, pupillometry, or hand gesture data. The responses to the questions associated with the digital content by the subject include at least one of: self-reported activity data, self-reported condition data, or patient responses to questionnaires and surveys. The predictive score indictive of a state of the subject can be generated using the trained machine learning model(s), based on the set of data streams. Depending on a percentage difference from a baseline and/or a measure about a predefined threshold, systems and devices described herein can be configured to predict a state of the subject based on the predictive score. The state of the subject includes a degree of brain plasticity or motivation for change of the subject. For example, if it is determined there is an increased brain plasticity or motivation for change, additional set of psychoeducational sessions can be provided to the subject based on the predictive score of the subject and historical data associated with the subject.

In some embodiments, systems and devices described herein can be configured to analyze patient data using a model or algorithm that can predict a current state of the patient's brain plasticity and/or motivation for change. The model or algorithm can produce a measure (e.g., an output) that represents current levels of the patient's brain plasticity and/or motivation for change. The measure can be compared to a measure of the patient's brain plasticity and/or motivation for change at an earlier time (e.g., a baseline) to determine whether the patient exhibits increased brain plasticity and/or motivation for change. In response to detecting a predetermined degree of increased brain plasticity and/or motivation (e.g., a predetermined percentage change or a measure above a predetermined threshold), systems and devices can generate and send an alert to a physician and/or therapist, at 618, and/or recommend timing for treatment, at 620. For example, after detecting that a patient has reached a predefined level of motivation, systems and devices can be configured to recommend to the physician and/or therapist to proceed with a drug treatment for the patient. Such can involve a method of treatment using a drug, therapy, etc., as further described below. If no increased brain plasticity and/or motivation is detected, systems and devices can return to providing additional assignments to the patient and/or terminate the digital therapy.

In some embodiments, systems and devices can be configured to predict potential adverse events for a patient, at 622. Examples of adverse events can include suicidal ideation, large mood swings, manic episodes, etc. In some embodiments, systems and devices described herein can predict adverse events by determining a significant change in a measure of a patient's mood. In some embodiments, the adverse event is a change in a measure of a patient's sleep patterns (such as a change in average sleep duration, number of times awakened per night). In some embodiments, the adverse event is a change in a measure of a patient's mood as determined by a clinical rating scale (such as the Short Opiate Withdrawal Scale of Gossop (SOWS-Gossop Hamilton Depression Rating Scale, the Clinical Global Impression (CGI) Scale, the Montgomery-Asberg Depression Rating Scale (MADRS), the Beck Depression Inventory (BDI), the Zung Self-Rating Depression Scale, the Raskin Depression Rating Scale, the Inventory of Depressive Symptomatology (IDS), the Quick Inventory of Depressive Symptomatology (QIDS), the Columbia-Suicide Severity Rating Scale, or the Suicidal Ideation Attributes Scale).

The HAM-D scale is a 17-item scale that measures depression severity before, during, or after treatment. The scoring is based on 17 items and generally takes 15-20 minutes to complete the interview and score the results. Eight items are scored on a 5-point scale, ranging from 0=not present to 4=severe. Nine items are scored on a 3-point scale, ranging from 0=not present to 2=severe. A score of 10-13 indicates mild depression, a score of 14-17 indicates mild to moderate depression, and a score over 17 indicates moderate to severe depression. In some embodiments, the adverse event is a change of a patient's mood as determined by an increases in the subject's HAM-D score by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

The MADRS scale is a 10-item scale that measures the core symptoms of depression. Nine of the items are based upon patient report, and 1 item is on the rater's observation during the rating interview. A score of 7 to 19 indicates mild depression, 20 to 34 indicates moderate depression, and over 34 indicates severe depression. MADRS items are rated on a 0-6 continuum with 0=no abnormality and 6=severe abnormality. In some embodiments, the adverse event is a change of a patient's mood as determined by an increases in the subject's MADRS score by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the adverse event is increase in one or more patient symptoms that indicate the patient is in acute withdrawal from drug dependence (such as sweating, racing heart, palpitations, muscle tension, tightness in the chest, difficulty breathing, tremor, nausea, vomiting, diarrhea, grand mal seizures, heart attacks, strokes, hallucinations and delirium tremens (DTs)).

In some embodiments, adverse events can be or be associated with one or more mental health or substance abuse disorders, including, for example, drug abuse or addition, a depressive disorder, or a posttraumatic stress disorder. For example, an adverse event can be an episode, an event, an incident, a measure, a symptom, etc. associated with a mental health or substance abuse disorder. In some embodiments, a mental health disorder or illness can be, for example, an anxiety disorder, a panic disorder, a phobia, an obsessive-compulsive disorder (OCD), a posttraumatic stress disorder, an attention deficient disorder (ADD, an attention deficit hyperactivity disorder (ADHD), a depressive disorder (e.g., major depression, persistent depressive disorder, bipolar disorder, peripartum or postpartum depression, or situation depression), or cognitive impairments (e.g., relating to age or disability).

In some implementations, systems and methods described herein can use software model(s) to generate a score or other measure of a patient's mood to generate periodic scores of a patient over time. The software model(s) can be, for example, an artificial intelligence (AI) model(s), a machine learning (ML) model(s), an analytical model(s), a rule based model(s), or a mathematical model(s). For example, systems and methods described herein can use a machine learning model or algorithm trained to generate a score or other measure of a patient's mood to generate periodic scores of a patient over time. In some implementations, machine learning model(s) can include: a general linear model, a neural network, a support vector machine (SVM), clustering, or combinations thereof. The machine learning model(s) can be constructed and trained using a training dataset. The training data set can include a historical dataset from a plurality of historical subjects. The historical dataset can include: biological data of the plurality of historical subjects, digital biomarker data of the plurality of historical subjects, and responses to questions associated with digital content by the plurality of historical subjects. The biological data of the plurality of historical subjects include at least one of: heart beat data, heart rate data, blood pressure data, body temperature, vocal-acoustic data, or electrocardiogram data. The digital biomarker data of the plurality of historical subjects includes at least one of: activity data, psychomotor data, response time data of responses to questions associated with the digital content, facial expression data, pupillometry, or hand gesture data. The responses to the questions associated with the digital content by the plurality of historical subjects include at least one of: self-reported activity data, self-reported condition data, or patient responses to questionnaires and surveys.

After the machine learning model(s) is trained using the training data, the systems and methods described in FIG. 6 steps 602, 604, 608, and 622 can be implemented using the trained machine learning model(s). For example, a set of data streams associated with the subject can be collected and using the trained machine learning model(s), a predictive score for the subject can be generated. Information can be extracted from the set of data streams that is being collected during a period of time before, during, or after administration of a drug to the subject. The set of data streams can include at least one of: biological data of the subject, digital biomarker data of the subject, or responses to questions associated with the digital content by the subject. The biological data of the subject include at least one of: heart beat data, heart rate data, blood pressure data, body temperature, vocal-acoustic data, or electrocardiogram data. The digital biomarker data of the subject includes at least one of: activity data, psychomotor data, response time data of responses to questions associated with the digital content, facial expression data, pupillometry, or hand gesture data. The responses to the questions associated with the digital content by the subject include at least one of: self-reported activity data, self-reported condition data, or patient responses to questionnaires and surveys. The predictive score for the subject can be generated using the trained machine learning model(s), based on the information extracted from the set of data streams. Depending on a percentage difference from a baseline and/or a measure about a predefined threshold, systems and devices described herein can be configured to predict whether an adverse event is likely to occur. Stated differently, a likelihood of an adverse event based on the predictive score can be determined.

Alternatively or additionally, systems and methods described herein can monitor for adverse events using a ruled based model(s), for example, using explicit questioning (e.g., “Do you have thoughts of injuring yourself?”) in a questionnaire or dialog. In response to predicting that an adverse event is likely to occur, systems and devices can generate and send an alert to a physician and/or therapist, at 624, and/or recommend content or treatment based on such detection, at 626. For example, systems and devices can be configured to recommend a change in content (e.g., a different series of assignments or a different type of content) to present to the patient, or recommend certain treatment or therapy for the patient (e.g., dosing strategy, timing for dosing and/or other therapeutic activities such as talk therapy, medication, check-ups, etc.), based on the analysis of the patient data. In some implementations, a drug therapy can be determined based on the likelihood of the adverse event. For example, in response to the likelihood of the adverse event being greater than a predefined threshold, a treatment routine for administrating a drug can be determined, based on historical data associated with the subject, and information indicative of a current state of the subject extracted from the set of data streams of the subject. The drug can include: ibogaine, noribogaine, psilocybin, psilocin, 3,4-Methylenedioxymethamphetamine (MDMA), N, N-dimethyltryptamine (DMT), or salvinorin A. If no adverse event is predicted, systems and devices can continue to provide additional assignments to the patient and/or terminate the digital therapy.

FIG. 7 depicts an example method 700 of analyzing patient data, according to embodiments described herein. Method 700 uses a machine learning model or algorithm (e.g., implemented by server 110, 210, 310 and/or machine learning system 254) to generate a predictive score or other assessment for evaluating a patient. For example, a processor executing instructions stored in memory associated with a machine learning system (e.g., machine learning system 254) or other compute device (e.g., server 110, 210, 310 or user device 120, 220, 320) can be configured to track information about a patient (e.g., mood, depression, anxiety, etc.).

In an embodiment, the processor can be configured to construct a model for generating a predictive score for a subject using a training dataset, at 702. The processor can receive patient data associated with a patient, e.g., collected during a period of time before, during, or after administration of a treatment of therapy to the patient, at 704. The processor can extract information corresponding to various parameters of interest from the patient data, at 706. The processor can generate, using the model, a predictive score for the subject based on the information extracted from the patient data, at 708. Such method 700 can be applied to analyze one or more different types of patient data, as described with reference to FIG. 6. The processor can further determine a state of the patient, e.g., based on the predictive score, by comparing the predictive score to a reference (e.g., a baseline), as described above with reference to FIG. 6.

2.4 Content Management

Content as described herein can be encoded into a normalized content format in a content creation application (e.g., content creation tool 252). The application can allow a content creator (e.g., a user) to create any of the content types described herein, including, for example, media-rich articles, videos, audio, surveys and questionnaires, and the like. Additionally, the application can allow the content creator to specify where in a content recursive content can appear and if certain content is to be blocked pending completion of other content. In some embodiments, the content creator can define how patient responses or interactions to content is interpreted by systems and devices described herein.

In some implementations, the application can cause digital content, for example, for a set of psychoeducational sessions to be stored and updated. The digital content file can include a set of digital features. The set of digital features can include at least one of: an interactive questionnaire or set of questions, a dialog activity, or embedded audio or visual content. When the creator creates a version of the digital content, metadata associated with the creation of the version of the digital content file is generated. The metadata can include: an identifier of the creator of the version of the digital content file, a time period or date associated with the creation, and a reason for the creation. Additionally, the version of the digital content file and the metadata associated with the version of the digital content file is hashed using a hash function to generate a pointer to the version of the digital content file. The version of the digital content that includes the pointer and the metadata associated with the version of the digital content file is saved in a content repository (e.g., content repository 242). When a user request to retrieve the version of the digital content file, the pointer is provided to the user. The version of the digital content file that includes the pointer, and the metadata associated with the version of the digital content file can be retrieved with the pointer. In some embodiments, such methods can be implemented using Git hash and associated functions.

In an embodiment, a content management system can include a system configured to encode content into a clear text format. The system can be implemented via a server (e.g., server 110, 210, 310), content repository (e.g., content repository 242), and/or content creation tool (e.g., content creation tool 252). The system can be configured to store the content in a version control system, e.g., on content repository. The system can be configured to track changes to the content and map changes to an author and/or reason for the change. The system can be configured to update, roll back or revert, and/or lock servers to a known state of the content. The system can be configured to encode rules for interpreting responses to content (e.g., responses to questionnaires and standardized instruments) into editable content, and to associate these rules with the applicable content or version of a digital content file including the applicable content.

In some embodiments, different versions of digital content can be created by one or more content creators. For example, a first content creator can create a first version of a digital content file, and a second content creator can modify that version of the digital content file to create a second version of a digital content file. A compute device implementing the content creation application can be configured to generate or create metadata associated with each of the first and second versions of the digital content file, and to store this metadata with the respective first and second versions of the digital content file. The compute device implementing the content creation application can also be configured to implement the hash function, e.g., to generate a pointer or hash to each version of the digital content file, as described above. In some embodiments, the compute device can be configured to send various versions of the digital content file to user devices (e.g., mobile devices of users such as a patient or a supporter) that can then be configured to present the digital features contained in the versions of the digital content file to the users. In some embodiments, the compute device can be configured to revert to older or earlier versions of a digital content file by reverting to sending the earlier versions of the digital content file to a user device such that the user device reverts back to presenting the earlier version of the digital content file to a user. In some embodiments, content creation can be managed by one creator or a plurality of creators, including a first, second, third, fourth, fifth, etc. creator.

2.5 Methods of Treatment

In some embodiments, systems and devices described herein can be configured to implement a method of treating a condition (e.g., mood disorder, substance use disorder, anxiety, depression, bipolar disorder, opioid use disorder) in a patient in need thereof. The method can include processing patient data (e.g., collected by a user device such as, for example, user device 120 or mobile device 220, 320) to determine a state of the patient, determining that the patient has a predefined mindset (e.g., brain plasticity or motivation for change) suitable for receiving a drug therapy based on the state of the patient or determining a likelihood of an adverse event, and in response to determining that the patient has the predefined mindset or there is a high likelihood of an adverse event, administering an effective amount of the drug therapy (e.g., ibogaine, noribogaine, psilocybin, psilocin, 3,4-Methylenedioxymethamphetamine (MDMA), N, N-dimethyltryptamine (DMT), or salvinorin A) to the subject to treat the condition.

In some embodiments, based on the mindset of a patient or the likelihood of an adverse event, the drug treatment or therapy can be varied or modified. For example, the dose of a drug (e.g., between about 1,000 μg to about 5,000 μg per day of salvinorin A or a derivative thereof, between about 0.01 to about 500 mg per day of ketamine, between about 20 mg to about 1000 mg per day or between about 1 mg to about 4 mg per kg body weight per day of ibogaine) can be varied depending the mindset of a patient or the likelihood of an adverse event. In some embodiments, a maintenance dose or additional dose may be administered to a patient, e.g., based on a patient's mindset before, during, or after the administration of the initial dose. In some embodiments, the dosing of a drug can be increased over time or decreased (e.g., tapered) over time, e.g., based on a patient's mindset before, during, or after the administration of the initial dose. In some embodiments, the administration of a drug treatment can be on a periodic basis, e.g., once daily, twice daily, three times daily, once every second day, once every third day, three times a week, twice a week, once a week, once a month, etc. In some embodiments, a patient can undergo long-term (e.g., one year or longer) treatment with maintenance doses of a drug. In some embodiments, dosing and/or timing of administration of a drug can be based on patient data, including, for example, biological data of the patient, digital biomarker data of the patient, or responses to questions associated with the digital content by the patient.

In some embodiments, systems and devices described herein can be configured to implement a method of treating a condition (e.g., mood disorder, substance use disorder, anxiety, depression, bipolar disorder, opioid use disorder) in a patient in need thereof. The method can include providing a set of psychoeducational sessions to a patient during a predetermined period of time preceding administration of a drug therapy to the subject, collecting patient data before, during, or after the predetermined period of time, processing the patient data to determine a state of the patient, identifying and providing an additional set of psychoeducational sessions to the subject based on the determined state, and administrating an effective amount of the drug, therapy, etc. to the subject to treat the condition.

In some embodiments, systems and devices described herein can be configured to process, after administering a drug, therapy, etc., additional patient data to detect one or more changes in the state of the subject indicative of a personality change or other change of the subject, a relapse of the condition, etc.

2.6 Questionnaire with Gesture Responses

Using the systems and techniques described herein, a questionnaire may be presented to a user, and the user may provide gesture-type responses. The system may further process the user's gestures. The server may present the questionnaire to the user's device, such as a mobile device. The user's device may include an interactive display, such as a touchscreen display. The server may transmit data corresponding to the questionnaire (e.g., questions) to the mobile device, where the data is received and displayed. An application (e.g., app) may be running on the user's device, and that application may process the data and present the questionnaire. The questionnaire may be displayed on a plurality of virtual pages (e.g., the same display displays different information for each “page”). For example, one or more questions may be presented on a first page, and when the answer(s) have been provided, then the next one or more questions is displayed on a subsequent page. When viewing a first virtual page, the user may make a gesture (e.g., touch-based gesture on touchscreen display), thereby providing an input signal. The input signal may be associated with a response to a given question on the questionnaire (e.g., a question presented on the first virtual page). The input signal may be processed (e.g., by the application, by the mobile device operating system, by the server, or a combination thereof). Processing may determine whether the input signal is a recognized gesture, and if so, the character of the gesture. Based on the character of the gesture, a value is assigned, which is then associated with an answer to the question. Subsequently, the process can be repeated for additional questions and virtual pages. The question(s) on the subsequent pages may vary based on responses to previous questions. Various examples of gestures and their associated values are described above.

A user may also respond to questions by providing a non-gesture input signal to the user's device. Such an input signal could come from a keyboard input, mouse input, or a non-gesture interaction with the touchscreen display—e.g., a tap on a screen to select a button.

While the questionnaire is being presented, the user's device (and/or another device) may gather additional information about the user and their response(s). For example, the time that it takes the user to answer a question could be measured. As another example, additional information may be received through a camera, such as sensing movement(s) of one or more parts of the user's body (e.g., head or eye movement(s)). As another example, the additional information could be received through biometric sensors (e.g., sensors for pulse, blood pressure, body temperature, or the like).

While various embodiments have been described above, it should be understood that they have been presented by way of example only, and not limitation. Where methods and/or schematics described above indicate certain events and/or flow patterns occurring in certain order, the ordering of certain events and/or flow patterns may be modified. While the embodiments have been particularly shown and described, it will be understood that various changes in form and details may be made.

Although various embodiments have been described as having particular features and/or combinations of components, other embodiments are possible having a combination of any features and/or components from any of embodiments as discussed above.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

Some embodiments and/or methods described herein can be performed by software (executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including C, C++, Java™ Ruby, Visual Basic™, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.), interpreted languages (JavaScript, typescript, Perl) or other suitable programming languages and/or development tools. Additional examples of computer code include, but are not limited to, control signals, encrypted code, and compressed code.

It will be understood by those skilled in the art that various changes may be made and equivalents may be substituted without departing from the scope of the novel techniques disclosed in this application. In addition, many modifications may be made to adapt a particular situation or material to the teachings of the novel techniques without departing from its scope. Therefore, it is intended that the novel techniques not be limited to the particular techniques disclosed, but that they will include all techniques falling within the scope of the appended claims.

1. A method of presenting and processing a digital questionnaire by a system including a server and a user's device executing an application, wherein the user's device includes an interactive display, the method comprising:
transmitting, from the server, data corresponding to the digital questionnaire to the user's device, wherein the digital questionnaire includes a plurality of virtual pages;
receiving, at the user's device, the data corresponding to the digital questionnaire from the server;
processing, by the application running on the user's device, the data corresponding to the digital questionnaire;
causing, by the application running on the user's device, data for a first one of the virtual pages to be presented on the interactive display of the user's device;
processing a first input signal corresponding to a first user input on the interactive display, wherein the first input signal is generated at least in part while the first one of the virtual pages is presented on the interactive display;
determining whether the first input signal includes data corresponding to a first gesture, wherein the first gesture is received at the interactive display;
determining a character of the first gesture;
assigning a first value corresponding to the first gesture;
assigning the first value as a response to a first question on the first one of the virtual pages;
causing, by the application running on the user's device, data for a second one of the virtual pages to be presented on the interactive display of the user's device;
processing a second input signal corresponding to a second user input on the interactive display, wherein the second input signal is generated at least in part while the second one of the virtual pages is presented on the interactive display;
determining the second input signal includes data corresponding to a second gesture, wherein the second gesture is received at the interactive display;
determining a character of the second gesture;
assigning a second value corresponding to the second gesture;
assigning the second value as a response to a second question on the second one of the virtual pages;
causing, by the application running on the user's device, data for a third one of the virtual pages to be presented on the interactive display of the user's device;
processing a third input signal corresponding to a third user input on the interactive display, wherein the third input signal is generated at least in part while the third one of the virtual pages is presented on the interactive display;
determining whether the third input signal includes data corresponding to a third gesture, wherein the third gesture is received at the interactive display;
determining a character of the third gesture; and
assigning a third value corresponding to the second gesture.

2. The method of claim 1, wherein the character of at least one of the first gesture, the second gesture, or the third gesture comprises a swipe.

3. The method of claim 1, wherein the at least one of the first value, the second value, or the third value comprises one of a binary value.

4. The method of claim 1, wherein at least one of the data for the second one of the virtual pages or the data for the third one of the virtual pages is selected based at least in part on at least one of the first value or the second value.

5. The method of claim 1, further comprising:
causing, by the application running on the user's device, data for a fourth one of the virtual pages to be presented on the interactive display of the user's device; and
processing a fourth input signal corresponding to a fourth user input on the interactive display, wherein the fourth input signal is generated at least in part while the fourth one of the virtual pages is presented on the interactive display,
wherein the fourth input signal does not include data corresponding to a gesture.

6. The method of claim 1, further comprising assessing at least one additional data during at least a portion of a time period between said causing data for the first one of the virtual pages to be presented on the interactive display of the user's device and said processing the first input signal corresponding to the first user input on the interactive display.

7. The method of claim 6, wherein the at least one additional data comprises at least one of a duration of the time period, a signal from a camera of the user's device, or biometric data.

8. A system for presenting a digital questionnaire to a user, the system comprising:
a user's device; and
a server, wherein:
the server is configured to transmit data corresponding to the digital questionnaire to the user's device, wherein the digital questionnaire includes a plurality of virtual pages;
the user's device is configured to receive the data corresponding to the digital questionnaire;
the user's device is configured to process the data corresponding to the digital questionnaire;
the user's device is configured to cause data for a first one of the virtual pages to be presented on the interactive display of the user's device;
at least one of the server or the user's device is configured to process a first input signal corresponding to a first user input on the interactive display, wherein the first input signal is generated at least in part while the first one of the virtual pages is presented on the interactive display;
at least one of the server or the user's device is configured to determine whether the first input signal includes data corresponding to a first gesture, wherein the first gesture is received at the interactive display;
at least one of the server or the user's device is configured to determine a character of the first gesture;
at least one of the server or the user's device is configured to assign a first value corresponding to the first gesture;
at least one of the server or the user's device is configured to assign the first value as a response to a first question on the first one of the virtual pages;
the user's device is configured to case data for a second one of the virtual pages to be presented on the interactive display of the user's device;
at least one of the server or the user's device is configured to process a second input signal corresponding to a second user input on the interactive display, wherein the second input signal is generated at least in part while the second one of the virtual pages is presented on the interactive display;
at least one of the server or the user's device is configured to determine the second input signal includes data corresponding to a second gesture, wherein the second gesture is received at the interactive display;
at least one of the server or the user's device is configured to determine a character of the second gesture;
at least one of the server or the user's device is configured to assign a second value corresponding to the second gesture;
at least one of the server or the user's device is configured to assign the second value as a response to a second question on the second one of the virtual pages;
the user's device is configured to cause data for a third one of the virtual pages to be presented on the interactive display of the user's device;
at least one of the server or the user's device is configured to process a third input signal corresponding to a third user input on the interactive display, wherein the third input signal is generated at least in part while the third one of the virtual pages is presented on the interactive display;
at least one of the server or the user's device is configured to determine whether the third input signal includes data corresponding to a third gesture, wherein the third gesture is received at the interactive display;
at least one of the server or the user's device is configured to determine a character of the third gesture; and
at least one of the server or the user's device is configured to assign a third value corresponding to the second gesture.

9. The system of claim 8, wherein the character of at least one of the first gesture, the second gesture, or the third gesture comprises a swipe.

10. The system of claim 8, wherein the at least one of the first value, the second value, or the third value comprises one of a binary value.

11. The system of claim 8, wherein at least one of the data for the second one of the virtual pages or the data for the third one of the virtual pages is selected based at least in part on at least one of the first value or the second value.

12. The system of claim 8, wherein:
the user's device is configured to cause data for a fourth one of the virtual pages to be presented on the interactive display of the user's device,
at least one of the server or the user's device is configured to process a fourth input signal corresponding to a fourth user input on the interactive display, wherein the fourth input signal is generated at least in part while the fourth one of the virtual pages is presented on the interactive display, and
wherein the fourth input signal does not include data corresponding to a gesture.

13. The system of claim 8, wherein at least one of the server or the user's device is configured to assess at least one additional data during at least a portion of a time period between said causing data for the first one of the virtual pages to be presented on the interactive display of the user's device and said processing the first input signal corresponding to the first user input on the interactive display.

14. The system of claim 13, wherein the at least one additional data comprises at least one of a duration of the time period, a signal from a camera of the user's device, or biometric data.

15. A non-transitory computer-readable storage medium comprising instructions that, when executed by at least one processor, cause the at least one processor to:
transmit, from a server, data corresponding to a digital questionnaire to a user's device, wherein the digital questionnaire includes a plurality of virtual pages;
receive, at the user's device, the data corresponding to the digital questionnaire from the server;
process, by the user's device, the data corresponding to the digital questionnaire;
cause, by the user's device, data for a first one of the virtual pages to be presented on the interactive display of the user's device;
process a first input signal corresponding to a first user input on the interactive display, wherein the first input signal is generated at least in part while the first one of the virtual pages is presented on the interactive display;
determine whether the first input signal includes data corresponding to a first gesture, wherein the first gesture is received at the interactive display;
determine a character of the first gesture;
assign a first value corresponding to the first gesture;
assign the first value as a response to a first question on the first one of the virtual pages;
cause, by the application running on the user's device, data for a second one of the virtual pages to be presented on the interactive display of the user's device;
process a second input signal corresponding to a second user input on the interactive display, wherein the second input signal is generated at least in part while the second one of the virtual pages is presented on the interactive display;
determine the second input signal includes data corresponding to a second gesture, wherein the second gesture is received at the interactive display;
determine a character of the second gesture;
assign a second value corresponding to the second gesture;
assign the second value as a response to a second question on the second one of the virtual pages;
cause, by the application running on the user's device, data for a third one of the virtual pages to be presented on the interactive display of the user's device;
process a third input signal corresponding to a third user input on the interactive display, wherein the third input signal is generated at least in part while the third one of the virtual pages is presented on the interactive display;
determine whether the third input signal includes data corresponding to a third gesture, wherein the third gesture is received at the interactive display;
determine a character of the third gesture; and
assigning a third value corresponding to the second gesture.

16. The non-transitory computer-readable storage medium of claim 15, wherein the character of at least one of the first gesture, the second gesture, or the third gesture comprises a swipe.

17. The non-transitory computer-readable storage medium of claim 15, wherein the at least one of the first value, the second value, or the third value comprises one of a binary value.

18. The non-transitory computer-readable storage medium of claim 15, wherein at least one of the data for the second one of the virtual pages or the data for the third one of the virtual pages is selected based at least in part on at least one of the first value or the second value.

19. The non-transitory computer-readable storage medium of claim 15, including further instructions that, when executed by at least one processor, further cause the at least one processor to:
cause data for a fourth one of the virtual pages to be presented on the interactive display of the user's device; and
process a fourth input signal corresponding to a fourth user input on the interactive display, wherein the fourth input signal is generated at least in part while the fourth one of the virtual pages is presented on the interactive display,
wherein the fourth input signal does not include data corresponding to a gesture.

20. The non-transitory computer-readable storage medium of claim 15, further comprising assessing at least one additional data during at least a portion of a time period between said causing data for the first one of the virtual pages to be presented on the interactive display of the user's device and said processing the first input signal corresponding to the first user input on the interactive display.
